(19) United States
(12) Patent Application Publication
(10) Pub. No.: US 2018/0177549 A1
(43) Pub. Date: Jun. 28, 2018

(54) AORTICORENAL GANGLION DETECTION

(71) Applicants: Douglas Christopher Harrington, Los Altos Hills, CA (US); Mark Thomas, Cupertino, CA (US); William David Holt, Los Gatos, CA (US)

(72) Inventors: Douglas Christopher Harrington, Los Altos Hills, CA (US); Mark Thomas, Cupertino, CA (US); William David Holt, Los Gatos, CA (US)

(21) Appl. No.: 15/885,652

(22) Filed: Jan. 31, 2018

Related U.S. Application Data
(63) Continuation of application No. 15/287,625, filed on Oct. 6, 2016.
(60) Provisional application No. 62/237,966, filed on Oct. 6, 2015.

(51) Int. Cl. A61B 18/14; A61B 18/18; A61B 18/24; A61N 1/36; A61B 18/12; A61B 18/00; A61N 7/00; A61N 7/02

(52) U.S. Cl. CPC A61B 18/1492; A61N 7/02; A61B 18/24; A61N 1/36; A61B 18/1206; A61B 2018/126; A61B 2018/1253; A61B 2018/00875; A61B 2018/00839; A61B 2018/00791; A61B 2018/00702; A61B 2018/00589; A61B 2018/00577; A61B 2018/00511; A61B 2018/00434; A61B 2018/00404; A61B 2018/00267; A61B 2018/0022; A61B 2018/00214; A61B 2018/0016; A61B 2018/1425; A61B 2018/143; A61B 2018/1435; A61N 2007/003; A61B 18/1815

(57) ABSTRACT

Devices and methods that regulate the innervation of the kidney by detection and modification of the aorticorenal ganglion. Devices for percutaneous detection and treatment of the aorticorenal ganglion via a blood vessel to modify renal sympathetic activity.

### CROSS-REFERENCE TO RELATED APPLICATIONS

This application claims priority to Provisional Application No. 62/237,966, filed Oct. 6, 2015 entitled Aorticorenal Ganglion Detection; and application Ser. No. 15/287,625, filed Oct. 6, 2016 entitled Aorticorenal Ganglion Detection, the entire contents of which are hereby incorporated by reference herein. This application is related to U.S. application Ser. No. 14/269,001, filed May 2, 2014 entitled Devices and Methods for Detection and Treatment of the Aorticorenal Ganglion, the entire contents of which are hereby incorporated by reference herein.

### BACKGROUND

Hypertension or abnormally high blood pressure is a growing public health concern for which successful treatment often remains elusive. Sixty-seven million Americans—about one-third of the adult population—have high blood pressure and these numbers are increasing as the population ages and obesity accelerates.

Hypertension is more common in men than women and afflicts approximately 50% of the population over the age of 65. Hypertension is serious because people with the condition have a higher risk for heart disease and other medical problems than people with normal blood pressure. If left untreated, hypertension can lead to arteriosclerosis, heart attack, stroke, enlarged heart and kidney damage.

Blood pressure is highest when the heart beats to push blood out into the arteries. When the heart relaxes to fill with blood again, the pressure is at its lowest point. Blood pressure when the heart beats is called systolic pressure. Blood pressure when the heart is at rest is called diastolic pressure. When blood pressure is measured, the systolic pressure is stated first and the diastolic pressure second. Blood pressure is measured in millimeters of mercury (mm Hg). For example, if a person's systolic pressure is 120 and diastolic pressure is 80, it is written as 120/80 mm Hg. Blood pressure lower than 120/80 mm Hg is considered normal.

A significant percentage of patients with uncontrolled hypertension fail to meet therapeutic targets despite taking multiple drug therapies at the highest tolerated doses, a phenomenon called resistant hypertension. This suggests there is an underlying pathophysiology resistant to current pharmacological approaches. Innovative therapeutic approaches are particularly relevant for these patients, as their condition puts them at high risk of major cardiovascular events.

The sympathetic nerve innervation of the kidney is implicated in the pathogenesis of hypertension through effects on rennin secretion, increased plasma rennin activity that leads to sodium and water retention, and reduction of renal (kidney) blood flow. As a result, a succession of therapeutic approaches has targeted the sympathetic nervous system to modulate hypertension, with varying success.

The sympathetic nerve innervation of the kidney is achieved through a dense network of postganglionic axons (nerves or nerve fibers) that innervate the kidney. This network of nerve fibers is often referred to as the renal plexus and runs alongside the renal artery and enters the hilum of the kidney. Thereafter, they divide into smaller nerve bundles following the blood vessels and penetrate cortical and juxtamedullary areas.

Preganglionic neuronal cell bodies are located in the intermediolateral cell column of the spinal cord. Preganglionic axons pass through the paravertebral ganglia (do not synapse) to become the lesser thoracic splanchnic nerve and least thoracic splanchnic nerve and travel to the aorticorenal ganglion (ARG) which is located at the origin of the renal artery from the abdominal aorta. Postganglionic axons then enter the renal plexus, where they play an important role in the regulation of blood pressure by effecting renin release. The renal plexus contains only sympathetic components. There is no (or at least very minimal) parasympathetic innervation of the kidney.

As a result of the renal sympathetic nerves being implicated in the pathophysiology of systemic hypertension, a succession of therapeutic approaches has targeted the sympathetic nervous system to modulate hypertension, with varying success.

Surgical sympathectomy, the surgical cutting of a sympathetic nerve, was attempted more than 40 years ago in patients with malignant hypertension. Malignant hypertension was a devastating disease with a five-year mortality rate of almost 100%, thus interventional approaches have been tested for its treatment given the lack of effective drug therapy at the time. Sympathectomy was mainly applied in patients with severe or malignant hypertension, as well as patients with cardiovascular deterioration despite relatively good blood pressure reduction by other means.

Sympathectomy, also termed splanchnicectomy, was performed either in one or two stages, required a prolonged hospital stay (2-4 weeks) and a long recovery period (1-2 months) and importantly had to be performed by a highly skilled surgeon. It was thus performed only in a few select centers in the U.S. and Europe.

Sympathectomy proved to be effective in reducing blood pressure immediately postoperatively, and the results were maintained in the long term in most patients. Survival rates were also demonstrated to be high for patients undergoing the procedure. The two major limitations of splanchnicectomy were the required surgical expertise and the frequent adverse events occurring with this procedure. Adverse events were common and included orthostatic hypotension (very low blood pressure when standing up), orthostatic tachycardia, palpitations, breathlessness, anhidrosis (lack of sweating), cold hands, intestinal disturbances, sexual dysfunction, thoracic duct injuries and atelectasis (collapse of the lung).

After the introduction of antihypertensive drugs and due to its poor patient tolerance and surgical difficulty, sympathectomy was reserved for patients who failed to respond to antihypertensive therapy or could not tolerate it.

Recent studies have focused on using thermal energy delivered through a percutaneous approach to achieve renal nerve denervation. Renal denervation performed this way is designed to damage the renal nerve fibers along the length of the artery using thermal energy to block renal nerve activity, thus neutralize the effect of the renal sympathetic system which is involved in the development of hypertension. Percutaneous thermal device based renal nerve denervation may achieve such objectives, but is limited to appropriate renovascular anatomy. For example, patients diagnosed with renal arteriogram are excluded from treatment with the Simplicity™ Renal Denervation System (Medtronic, Minneapolis, Minn.) if renal artery diameter is less than 4 mm or renal artery length is less than 20 mm. Patients with accessory renal arteries, approximately 20-30% of the patient population, are also excluded from treatment.

Renal nerve denervation has also raised concerns of complications arising from significant amount of thermal endothelial damage required to create a complete renal nerve block along the length of the renal artery. Cases of renal artery stenosis after thermal renal nerve denervation have been reported in the literature.

As described above, the aorticorenal ganglion plays an import role in renal function including blood pressure regulation. Maillet (Innervation sympathique du rein: son role trophique. Acta Neuroveg., Part II, 20:337-371, 1960) describes various lesions of the renal parenchyma (functional tissue of the kidney, including the nephrons) after the chemical destruction of the aorticorenal ganglion in an animal model. Carbolic acid (5%) was brushed on the left aorticorenal ganglion or the left renal plexus. The renal parenchyma changes between the two techniques were shown to be identical.

Dolezel (Monoaminergic innervation of the kidney. Aorticorenal ganglion—a sympathetic, monoaminergic ganglion supplying the renal vessels. Experientia, 23:109-111, 1967) extirpated the left aorticorenal ganglion from 8 canines. 6-8 days later the left kidney was harvested and examined. Throughout the whole kidney the monoaminergic nerves terminating on the surface of the media of arteries, on the vasa recta, on the veins, in the fibrous skeleton of the kidney, and in the muscular part of the pelvic wall showed complete degeneration.

Norvell (Aorticorenal ganglion and its role in renal innervation. J. Comp. Neural., 133:101-111, 1968) describes removing the aorticorenal ganglia from one side of 14 adult felines. Two weeks later, the kidneys were harvested and examined. Norvell observed that the large bundle of nerve fibers which are normally present in the perivascular connective tissue of the control kidney were found less frequently in the experimental kidneys. In the control kidneys, at least one, and sometimes several bundles of nerve fibers, was associated with any large blood vessel observed under the microscope. This was not the case in the experimental kidneys. It was difficult to find even a small bundle of nerve fibers in the area around the blood vessels. Fine nerve fibers going to the tubules were even more difficult to locate. Norvell concluded from the reduction of nerve fibers seen in the cat after removal of the aorticorenal ganglion, that this ganglion is important in both tubular and vascular innervation.

Various animal studies have shown that electrically stimulating renal nerves influences changes in renal hemodynamics such as renal blood flow (RBF) and glomerular filtration rate (GFR). From these animal studies emerged the concept of the graded response of the renal neuroeffectors to graded increase in the frequency of renal sympathetic nerve stimulation. At the lower frequency range (≈0.5 Hz), there is stimulation of renin secretion rate (RSR), without effects on urinary sodium excretion (U_{NA}V), RBF or GFR. At slightly higher frequencies (≈1.0 Hz), there is both stimulation of RSR and a decrease in UNAV, without effects on RBF or GFR. At higher frequencies (≈2.0 Hz), there is stimulation of RSR and a decrease in U_{NA}V and renal vasoconstriction, with decrease RBF (Gerald F. DiBona, Neural Control of the Kidney Past, Present and Future, Hypertension 2003; 41 [part 2]:621-624).

There is the need for a method and device that can regulate the innervation of the kidney to control diseases related to kidney function including hypertension without the limitations associated with only targeting renal nerve fibers with thermal energy.

### SUMMARY

The invention relates to devices and methods for treating hypertension and its related conditions. The method includes percutaneous modification of the aorticorenal ganglion and/or postganglionic renal nerves which results in a decrease or cessation of kidney nerve activity involved in the development of hypertension. The method can include but is not limited to the use of thermal, cryogenic, electrical, chemical, radiation, pharmacological and mechanical techniques to modify or neutralize the ganglion by means of a catheter.

Embodiments of the present invention are directed to a catheter assembly including a tissue modifying element or elements located approximately at the distal end of said catheter. One method involves percutaneous placement of the catheter in the renal artery in proximity to the aorticorenal ganglion followed by activation of the tissue modifying element. Activation modifies (e.g. ablates when radiofrequency energy is employed) the ganglion, creating disruption of nerve signals leading to the kidney. Other methods involve percutaneous placement of the catheter in any of the other body lumens in proximity to the aorticorenal ganglion including but not limited to abdominal aorta, vena cava, renal vein and ostia.

In accordance with an aspect of the current invention, an aorticorenal ganglion modifying catheter comprises an elongated catheter body extending longitudinally between a proximal end and a distal end along a longitudinal axis and a tissue modifying element or elements attached to the catheter body, elements to be utilized by activation which results in ganglionic tissue modification. One embodiment of the present invention is directed to a catheter assembly including a single monopolar radiofrequency electrode element located at the distal end of said catheter. In this embodiment the proximal end of the catheter is connected to an electrosurgical generator which in turn is connected to a dispersive electrode pad attached to the patient's skin creating a closed electrical circuit when electrode element is in tissue contact. When activated, radiofrequency energy travels through tissue adjacent to electrode and heats tissue resulting in tissue ablation and modification of the aorticorenal ganglion.

Another embodiment of the present invention is directed to a catheter assembly including a multi-electrode bipolar radiofrequency electrode element located at the distal end of said catheter. Use is similar to monopolar catheter but does not require the use of dispersive electrode pad. Another embodiment of the present invention is directed to radiofrequency electrode element with a cooling feature at the distal end of said catheter. Cooling the RF electrode element during activation has several benefits including limiting endothelial tissue damage to the vessel wall and creating deeper tissue modification (e.g. deeper lesions) if desired. Cooling the RF electrode element allows for higher temperatures thus deeper lesions by preventing high impedance electrosurgical generator shut down which occurs when blood coagulation collects on the higher temperature electrode elements. Cooling mechanism may incorporate a peltier effect device, cooled fluid or gas circulated in catheter distal tip. One example of cooling the electrode element involves flushing saline through catheter body and out of through-holes manufactured into the electrode element into the blood stream during radiofrequency energy activation, thus cooling the hotter electrode element with the cooler fluid through heat transfer.

In accordance with an aspect of the current invention, an aorticorenal ganglion modifying catheter comprises an elongated catheter body extending longitudinally between a proximal end and a distal end along a longitudinal axis and a balloon element assembly connected to the catheter body comprising radiofrequency electrode element attached to outer surface of balloon element. Balloon element has a proximal end connected to catheter body and a distal end. Balloon element is movable between a collapsed configuration and an expanded configuration. When balloon element is in proximity of aorticorenal ganglion, balloon element is expanded allowing for tissue contact with radiofrequency electrode element. Ganglionic tissue modification is achieved as previously described with monopolar and bipolar electrode element catheters.

In accordance with an aspect of the current invention, an aorticorenal ganglion modifying catheter comprises an elongated catheter body extending longitudinally between a proximal end and a distal end along a longitudinal axis and a basket element assembly connected to the catheter body comprising radiofrequency electrode elements attached to outer surface of basket element. Basket element has a proximal end connected to catheter body and a distal end. Basket element is movable between a collapsed configuration and an expanded configuration. When basket element is in proximity of aorticorenal ganglion, basket element is expanded allowing for tissue contact with radiofrequency electrode element. Ganglionic tissue modification is achieved as previously described with monopolar and bipolar electrode element catheters.

In accordance with an aspect of the current invention, an aorticorenal ganglion modifying catheter comprises an elongated catheter body extending longitudinally between a proximal end and a distal end along a longitudinal axis and a coil element assembly connected to the catheter body comprising radiofrequency electrode elements attached to surface of coil element. Coil element has a proximal end connected to catheter body and a distal end. Coil element is movable between a collapsed configuration and an expanded configuration. When coil element is in proximity of aorticorenal ganglion, coil element is expanded allowing for tissue contact with radiofrequency electrode element. Ganglionic tissue modification is achieved as previously described with monopolar and bipolar electrode element catheters.

In accordance with an aspect of the current invention, an aorticorenal ganglion modifying catheter comprises an elongated catheter body extending longitudinally between a proximal end and a distal end along a longitudinal axis and a radiofrequency electrode needle element contained within the catheter body. Radiofrequency electrode element can comprise either a monopolar or bipolar design and is movable between a retracted arrangement and a slidably advanced arrangement. One method involves percutaneous placement of the catheter in proximity to the aorticorenal ganglion, advancement of the radiofrequency electrode needle element through the vessel wall in juxtaposition to or within ganglion followed by activation of the tissue modifying electrode needle. Ganglionic tissue modification is achieved as previously described with monopolar and bipolar electrode element catheters.

Anatomically, the aorticorenal ganglion may be located just superior, anterior or inferior to the renal artery. One method of treatment involves creating tissue modification (e.g. tissue ablation when radiofrequency energy is employed) in the anatomic regions associated with the location of the aorticorenal ganglion. The shape of such a lesion would generally resemble a half toroid or half doughnut or horseshoe shaped tissue modification zone. Lesion shape can be contiguous or contain discrete segments that generally look similar to a half toroid in shape.

Half toroid shaped lesions can be created with previously disclosed embodiments of the current invention or with various design modifications of the previously disclosed embodiments. One method involves percutaneous placement and treatment with the monopolar radiofrequency aorticorenal ganglion modifying catheter in discrete segments along the vessel. For example, radiofrequency electrode element can be repositioned for tissue contact and activated in a superior, anterior and inferior position with the renal artery adjacent the ostium. Shape of tissue modification (e.g. lesion) will generally look similar to a half toroid.

Aorticorenal ganglion modifying catheters comprising either a balloon element, basket element or coil element previously disclosed can also be modified to create a half toroid shape lesions by bias positioning of the radiofrequency electrode elements. For example, electrode elements may be positioned on superior, anterior and inferior surface of balloon, basket or coil. One method involves placement of modified balloon, basket or coil catheter within renal artery so that tissue contact with electrode elements is superior, anterior and inferior to renal artery when balloon, basket or coil are expanded, followed by activation of the tissue modifying electrodes as previously described.

Aorticorenal ganglion modifying catheter comprising a radiofrequency electrode needle element previously disclosed can also be modified to create a half toroid shape lesion by biased positioning of more than one electrode needle element. For example, two or more electrode needle elements may be attached to the superior, anterior and inferior catheter body. One method involves placement of modified multi-needle element catheter within renal artery so that advancement of radiofrequency electrode needle elements through vessel wall is superior, anterior and inferior to renal artery, followed by activation of the tissue modifying needle electrodes as previously described.

In animal models, the aorticorenal ganglion has been located between the renal artery and renal vein. One method of treatment involves percutaneous placement of aorticorenal ganglion modifying catheter into the renal vein for modification of the aorticorenal ganglion.

Present invention also relates to devices and methods for detection of the aorticorenal ganglion by stimulating the aorticorenal ganglion and measuring resulting physiological responses. Examples of electrically stimulated physiological responses detectable at approximately 2 to 20 Hz stimulation include renal vasoconstriction, decreased RBF, decreased GFR and kidney and renal vasculature pulsations. Electrical stimulation can also be applied at approximately 50 Hz to stimulate sensory (afferent) nerves resulting in patient sensation and feedback to the medical staff. Detection method using electrical stimulation includes percutaneous placement of a tissue stimulating radiofrequency catheter with distal tip electrode in the renal vasculature adjacent to the aorticorenal ganglion followed by delivery of electrical energy (e.g. 15 volts, 5 Hz, 0.500 msec. pulse duration) through said tip into vessel wall. Stimulation of the ganglion will cause a detectable physiological response such as renal vasoconstriction, decreased RBF, reduced GFR and pulsations of the kidney and renal vasculature.

Renal vasoconstriction caused by electrical stimulation of the ganglion may be evaluated by measuring the change in renal artery diameter with diagnostic technologies such as Magnetic Resonance Angiogram (MRA), Angiography, Sonography (ultrasound), intravascular ultrasound (IVUS) (e.g. Eagle Eye® Platinum Catheter, Volcano Corporation, San Diego Calif.), and Optical Coherence Tomography (OCT) (e.g. Dragonfly™ Duo OCT Imaging Catheter, St. Jude Medical, St. Paul, Minn.). Vasoconstriction may also be evaluated with tissue stimulating catheter embodying a balloon, basket, coil or the like element by measuring the change in radial dimensions of the element during stimulation. For example, balloon element with radiopaque markers attached to the surface of a compliant balloon and placed within renal vessel will radially converge, as observed under fluoroscopy, during ganglia or nerve stimulation. Element may also transmit compression data (in the form of pressure increase for balloon element with pressure transducer embodiment) to an external source for vasoconstriction assessment.

Change in renal blood flow caused by electrical stimulation of the ganglion may be evaluated directly and/or indirectly with diagnostic technologies such as external Doppler Sonography and Intravascular Doppler Sonography which measures blood flow velocity (e.g. FloWire® Doppler Guide Wire, Volcano Corporation, San Diego Calif.) and Thermal Dilution Catheter which measures blood flow (e.g. Swan-Ganz catheter, Edwards Life Science, Irvine Calif.).

Kidney and renal artery pulsations caused by electrical stimulation of the ganglion may be visualized and evaluated with diagnostic technologies such as Magnetic Resonance Angiography (MRA), Angiography, Sonography and Doppler Sonography.

Kidney and renal artery pulsations caused by electrical stimulation of the ganglion which creates blood pressure pulsations in the renal artery may be evaluated with diagnostic technologies such as Intravascular Pressure Wire which measures blood pressure (e.g. Verrata™ Pressure Guide Wire, Volcano Corporation, San Diego Calif.).

Tissue stimulating device and/or physiological measurement device (diagnostic technologies) can be incorporated as elements into aorticorenal ganglion modifying catheter. Tissue stimulating element may also be used as tissue modifying element, for example when metallic electrodes are used for electrical stimulation and radiofrequency ablation. Tissue stimulating element may incorporate one or multiple distal tip electrodes and can be designed as a basket electrode, coil electrode, balloon electrode or the like and as previously disclosed in embodiments of the aorticorenal ganglion modifying catheter.

Procedure steps for detection, modification and treatment verification of aorticorenal ganglion or other targeted nerve tissue may be as follows: Step 1, locate aorticorenal ganglion by applying stimulation and analyzing a physiological response. Step 2, modification of aorticorenal ganglion (e.g. tissue ablation with RF energy). Step 3 (optional), confirmation of adequate modification of aorticorenal ganglion by reapplying stimulation and analyzing the physiological response.

Embodiments of the present invention are directed to a catheter assembly including a tissue stimulating element and a tissue modifying element located approximately at the distal end of said catheter. Stimulating element and tissue modifying element may be integral or separate components. One method of detection and modification of the aorticorenal ganglion involves percutaneous placement of said catheter in the renal artery with stimulating element and modifying element adjacent the vessel wall followed by electrical stimulation of adjacent tissue with stimulating element. Ganglion location is determined with a measurable or observable physiological response (e.g. renal vasoconstriction as detected during fluoroscopy). Modification of the ganglion then proceeds (e.g. ablation when radiofrequency energy is employed) by activation of the tissue modifying element adjacent stimulated tissue, resulting in disruption of the nerve signals leading to the kidney. Sufficient ganglion treatment may be confirmed by reapplying electrical stimulation to modified tissue and discerning differences to the pre-treatment physiological response. Method of detection and modification with said catheter may also be performed in other vessels including the renal vein, vena cava or aorta.

Embodiments of the present invention are also directed to a catheter assembly including a tissue modifying element and a physiological measurement element located approximately at the distal end of said catheter. One method of modification of the aorticorenal ganglion involves percutaneous placement of said catheter in the renal artery followed by baseline physiological measurements with physiological measurement element. Modification of the ganglion then proceeds by activation of the tissue modifying element, resulting in disruption of the nerve signals leading to the kidney. Acceptable nerve signal disruption may be confirmed by comparing the differences between pre-tissue modification physiological responses to post-tissue modification physiological responses with said catheter. Modification of the ganglion and physiological response measurements may be performed separately or simultaneously, with the latter allowing for a cessation of tissue modification once acceptable nerve disruption as measured by a physiological response is achieved.

Another embodiment of the present invention is directed to a catheter assembly including a tissue stimulating element, physiological measurement element and tissue modifying element located approximately at the distal end of said catheter. Tissue stimulating element, physiological measurement element and tissue modifying element may be integral or separate components of said catheter. One method of detection and modification of the aorticorenal ganglion involves percutaneous placement of the catheter in the renal artery with stimulating element and tissue modifying element adjacent the vessel wall and physiological measurement element proximate aforementioned elements. Electrical stimulation is applied with tissue stimulating element to adjacent tissue followed by measurement of a response to the stimuli with the physiological measurement element. Ganglia detection is confirmed when pre-established physiological response measurements for targeted ganglion are achieved. Modification of the ganglion then proceeds (e.g. ablation when radiofrequency energy is employed) by activation of the tissue modifying element adjacent stimulated tissue, resulting in disruption of the nerve signals leading to the kidney. Adequate ganglion treatment may be confirmed by reapplying electrical stimulation to modified tissue and comparing differences to the pre-treatment physiological response. Method of detection and modification of ganglia and nerve tissue with said catheter may also be performed within other vessels including the renal vein, vena cava or aorta. Present invention may also target and treat alternative ganglia, splanchnic nerves and the renal plexus.

Embodiments of the present invention are also directed to a two catheter arrangement embodying a tissue stimulating element, tissue modifying element and physiological measurement element. One arrangement including a first catheter with a tissue stimulating element and tissue modifying element at the distal end of said first catheter and a second catheter with a physiological measurement element at the distal end of said second catheter. An alternative arrangement including a first catheter with a tissue stimulating element and physiological measurement element at the distal end of said first catheter and second catheter with a tissue modifying element at the distal end of said second catheter. An alternative arrangement including a first catheter with a tissue modifying element and physiological measurement element at the distal end of said first catheter and second catheter with a tissue stimulating element at the distal end of said second catheter. One method of detection and modification of the aorticorenal ganglion with said two catheter arrangement involves percutaneous placement of first catheter with tissue stimulating element and tissue modifying element in the renal vein and percutaneous placement of second catheter with physiological measurement element in the renal artery. Electrical stimulation of adjacent tissue is applied with said first catheter and physiological response is ascertained with said second catheter to locate the ganglion as described previously. Modification of the ganglion then proceeds by activation of the tissue modifying element on said first catheter followed by verification of treatment by reapplying stimulation to modified tissue with said first catheter and analyzing physiological responses with said second catheter. Methods of treatment with two catheter arrangements may also be performed in various combinations of placement of devices in the renal vein, renal artery, vena cava and aorta. For example, placement of first catheter with a tissue stimulating element and tissue modifying element placed in the vena cava and second catheter with physiological measurement element placed in the renal artery.

Embodiments of the present invention are also directed to a two catheter arrangement embodying a tissue stimulating element and tissue modifying element. One said arrangement including a first catheter with a tissue stimulating element at the distal end of said first catheter and second catheter with a tissue modifying element at the distal end of said second catheter. One method of detection and modification of the aorticorenal ganglion with said two catheter arrangement involves percutaneous placement of the first catheter in the renal vein with stimulating element and percutaneous placement of second catheter with tissue modifying element in the aforementioned renal vein. First catheter delivers electrical stimulation to adjacent tissue followed by measurement of a physiological response (e.g. renal vasoconstriction as detected during fluoroscopy) to locate the ganglion. Modification of the ganglion then proceeds with second catheter by activation of the tissue modifying element adjacent tissue (e.g. ablation when high intensity focused ultrasound is employed), resulting in disruption of the nerve signals leading to the kidney. Sufficient ganglion treatment may be confirmed by reapplying electrical stimulation to modified tissue and analyzing differences to the pre-treatment physiological response. Method of nerve detection and tissue modification with said two catheter arrangement may also be performed in various combinations within the renal veins, arteries, vena cava and aorta.

Embodiments of the present invention are also directed to a three catheter arrangement. One said arrangement including a first catheter with a tissue stimulating element at the distal end of said first catheter, second catheter with a physiological measurement element at the distal end of said second catheter and third catheter with a tissue modifying element at the distal end of said third catheter. Method of detection and modification of ganglia with three catheter arrangement is similar to the previously described procedures with percutaneous placement of said catheters in the renal veins, arteries, vena cava and aorta. For example, one method involves percutaneous placement of the first catheter with tissue stimulating element in the aorta, second catheter with physiological measurement element in the renal artery and third catheter with tissue modifying element in the renal vein. Electrical stimulation is applied to the adjacent tissue in the aorta with first catheter, stimulating the splanchnic nerve followed by measurement of a physiological response in the renal artery with second catheter. Renal innervation of the stimulated nerve is confirmed when physiological response (e.g. renal artery diameter contraction) is detected with said second catheter. Modification of the nerve tissue then proceeds by activation of the tissue modifying element with said third catheter. Verification of nerve treatment may be confirmed by reapplying electrical stimulation with said first catheter and analyzing physiological response changes with said second catheter.

Percutaneous placement of the catheter assembly may be accomplished using any of the currently available techniques and ancillary equipment for abdominal aorta and renal artery interventions including guided sheaths, steerable distal tip assemblies and over the wire configurations employed for diagnostic and therapeutic devices. There may be other means to modify the aorticorenal ganglion not specifically described in one of the inventions embodiments, but it is to be understood that the description is not meant as a limitation since further modifications may suggest themselves or be apparent to those skilled in the art.

The invention disclosed herein may be utilized for treatment of other clinical conditions influenced by kidney nerve activity including kidney disease, congestive heart failure, obstructive sleep apnea, diabetes and others. The invention disclosed herein may be utilized for modification of other tissues including splanchnic nerves, renal nerves and ganglia apart from aorticorenal ganglia.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is an anterior view of human kidneys and supporting vasculature;

FIG. 2 is a posterior view of human kidneys and supporting vasculature;

FIG. 3 is an anterior view of the innervation of the right kidney;

FIGS. 4a and 4b are schematic views of a radiofrequency energy aorticorenal ganglion modifying system;

FIG. 5 is a close up view of the monopolar aorticorenal ganglion modifying catheter located in the renal artery;

FIG. 6a-6c is a close up view of the balloon aorticorenal ganglion modifying catheter located in the renal artery;

FIG. 7a-7c is a close up view of the basket aorticorenal ganglion modifying catheter located in the renal artery;

FIGS. 8a and 8b is a close up view of the needle electrode aorticorenal ganglion modifying catheter located in the renal artery;

FIGS. 9a and 9b is an anterior and sagittal view of the right aorticorenal ganglion;

FIGS. 10a and 10b is an anterior and sagittal view of the right aorticorenal ganglion contained within a tissue modification zone;

FIG. 11a-11c is a schematic of the balloon aorticorenal ganglion detection and modifying system;

FIGS. 12a and 12b are schematic views of the basket aorticorenal ganglion detection and modifying system;

FIG. 13 is a schematic of the coil aorticorenal ganglion detection and modifying system;

FIGS. 14A and 14B are a schematic view of an aorticorenal ganglion detection and modifying system;

FIGS. 15A and 15B are a schematic view of an aorticorenal ganglion detection and modifying system;

FIG. 16 a schematic view of an aorticorenal ganglion detection and modifying system;

FIG. 17 is a frame capture of a baseline nephrogram;

FIG. 18 is a frame capture of a nephrogram performed with stimulation before tissue modification;

FIG. 19 is a frame capture of a nephrogram performed with stimulation after tissue modification;

FIGS. 20a and 20b are schematic views of an aorticorenal ganglion detection and modifying system;

FIGS. 21a and 21b are schematic views of an aorticorenal ganglion detection and modifying system;

FIG. 22 is a schematic view of an aorticorenal ganglion detection and modifying system;

FIG. 23 is a schematic view of an aorticorenal ganglion detection and modifying system;

FIG. 24 is a schematic view of an amplifier for an aorticorenal ganglion detection and modifying system;

FIG. 25 is a schematic view of an aorticorenal ganglion detection and modifying system;

FIG. 26 is a schematic view of an aorticorenal ganglion detection and modifying system;

FIGS. 27a and 27b are schematic views of an aorticorenal ganglion detection and modifying system;

FIG. 28 is a schematic view of an aorticorenal ganglion detection and modifying system;

FIGS. 29a-29c are schematic views of an aorticorenal ganglion detection and modifying system;

FIGS. 30a-30f are schematic views of an aorticorenal ganglion detection and modifying system;

FIG. 31 is a schematic view of an aorticorenal ganglion detection and modifying system;

FIG. 32 is a schematic view of an aorticorenal ganglion detection and modifying system;

FIGS. 33a-33b are schematic views of a lensing system for an aorticorenal ganglion detection and modifying system;

FIG. 34 is a schematic view of an aorticorenal ganglion detection and modifying system;

FIGS. 35a-35b are schematic views of an ultrasound system for an aorticorenal ganglion detection and modifying system;

FIGS. 36a-36b are schematic views of an aorticorenal ganglion detection and modifying system;

FIG. 37 is a schematic view of an aorticorenal ganglion detection and modifying system;

FIGS. 38-40 are flow charts for a method of operation of an aorticorenal ganglion detection and modifying system;

FIGS. 41-42 are schematic views of an aorticorenal ganglion detection and modifying system; and,

FIGS. 43a-43b are schematic views of an aorticorenal ganglion detection and modifying system.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

U.S. application Ser. No. 14/269,001 is directed to additional devices, techniques, and methods for treatment of various conditions, such as high blood pressure, via the ARG. This application is incorporated herein by reference in its entirety.

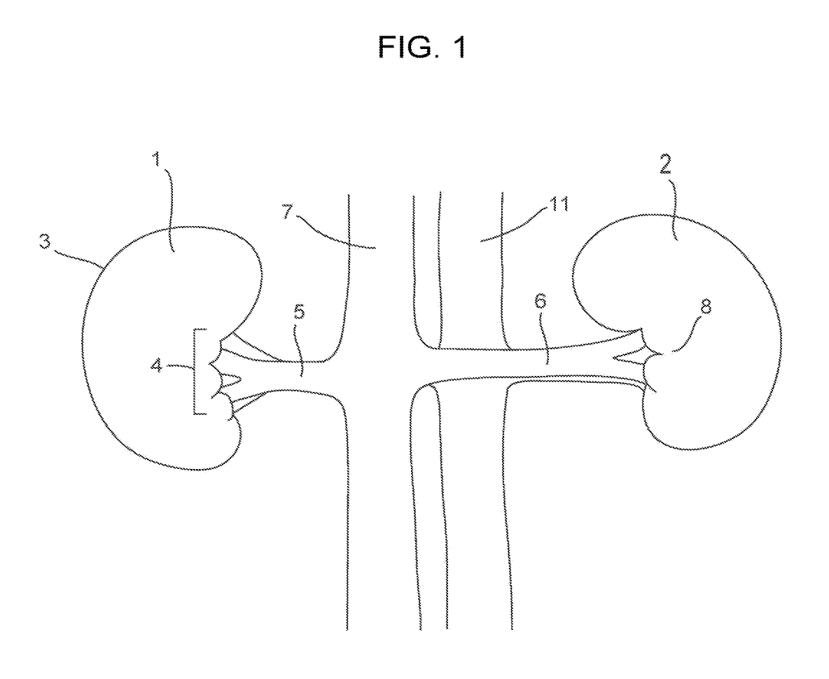

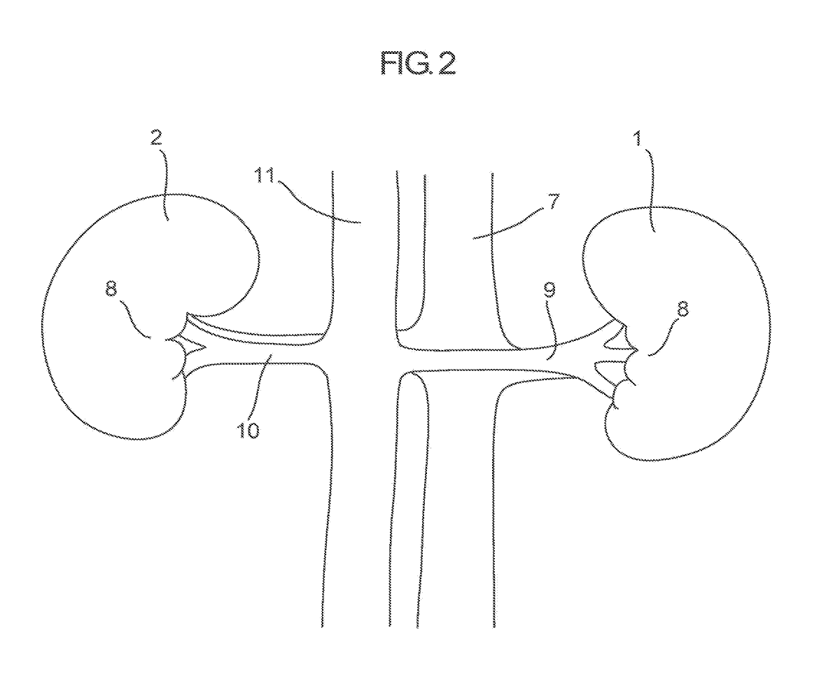

FIG. 1 is an anterior view illustration of the kidneys and major arteries and veins supporting the kidneys. The right kidney 1 and left kidney 2 are bean-shaped organs, each approximately the size of a tightly clenched fist. They lie on the posterior abdominal wall behind the peritoneum and on either side of the vertebral column while the superior pole of each kidney is protected by the rib cage. A fibrous connective tissue renal capsule 3 surrounds each kidney and around the capsule is a dense deposit of adipose tissue, the renal fat pad (not shown), which protects the kidney and supporting vasculature. On the medial side of each kidney is a relatively small area called the hilum 4 where the renal artery and the nerves enter and the renal vein and the ureter (not shown) exit. The right renal vein 5 and left renal vein 6 branches off the inferior vena cava 7 and enters the renal sinus 8 of each kidney. Renal veins are blood vessels that carry deoxygenated blood out of the kidney to the inferior vena cava 7. FIG. 2 is a posterior view illustration of the kidneys and major arteries and veins supporting the kidneys. The right renal artery 9 and left renal artery 10 branches off the abdominal aorta 11 and enter the renal sinus 8 of each kidney. The renal arteries carry a large portion of total blood flow to the kidneys. Up to a third of total cardiac output can pass through the renal arteries to be filtered by the kidneys.

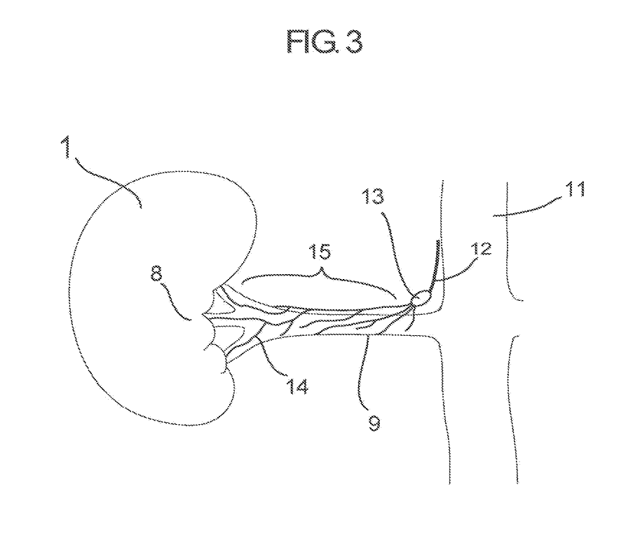

FIG. 3 is an anterior view illustration of the right kidney 1 and right renal artery 9 with the renal vein and inferior vena cava removed. The lesser and least thoracic splanchnic nerves 12 originate in the spinal cord and travel to the aorticorenal ganglion 13 which is located at the origin of the renal artery 9 from the abdominal aorta 11. Postganglionic axons 14 then form the renal plexus 15, as this dense network of nerve fibers is often referred to, which runs alongside the renal artery and enters the hilum 4 of the kidney 1. Thereafter, they divide into smaller nerve bundles following the blood vessels and penetrate cortical and juxtamedullary areas.

A ganglion is typically known as a mass of tissue formed by ganglion cells. Ganglia can provide relay points and intermediary connections between different neurological structures in the body, such as the peripheral and central nervous systems. There is typically one aorticorenal ganglion 13 for each renal plexus (2 per human) and it can be located superior, anterior and inferior to the renal artery. Its size can vary from a small swelling approximately 1 mm in diameter to an irregular shape approximately 10 mm long and 5 mm wide.

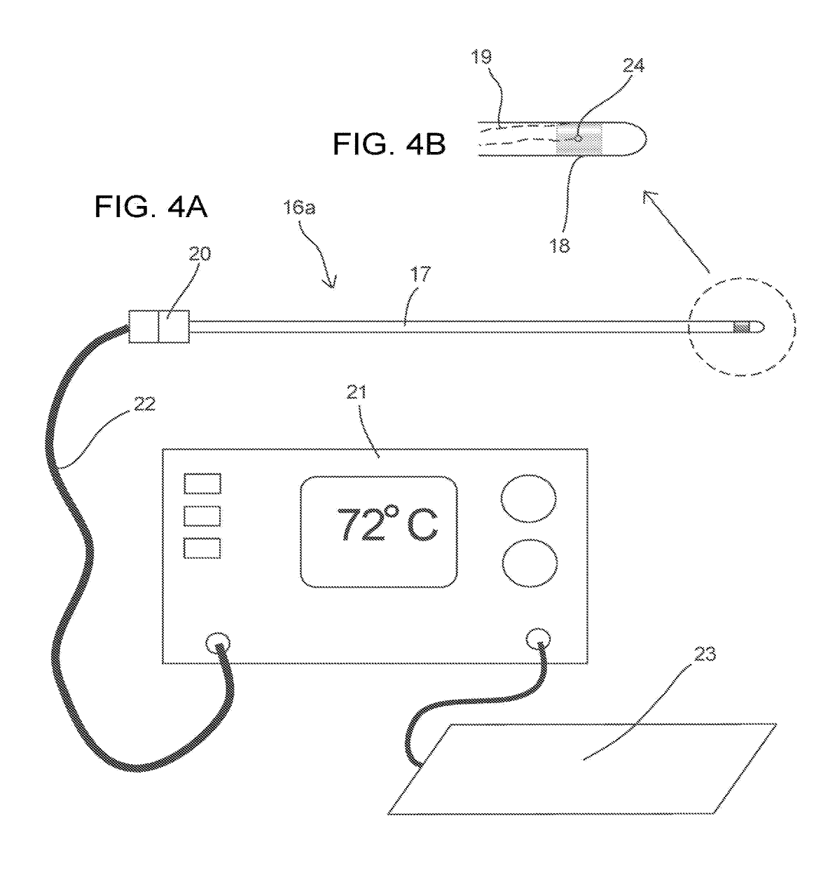

Percutaneous aorticorenal ganglion modification may be accomplished by delivery of energy to a tissue modifying element located at the distal end of the catheter using an external energy source. Transmission of the energy to the tissue modifying element may be accomplished by various means including transmission through an energy transmitting conduit located within a catheter body that extends the length to the proximal end of the catheter body. Proximal end of catheter body may be coupled by way of connectors and/or cables to external energy source. For example, FIG. 4A is a schematic of an aorticorenal ganglion modifying system utilizing radiofrequency energy. Aorticorenal ganglion modifying catheter 16a comprises an elongated body 17 extending longitudinally between a proximal end and a distal end along a longitudinal axis and comprising an electrode as the tissue modify element 18 located approximately at the distal end of the catheter. Tissue modifying element 18 utilizing an electric current for operation can be manufactured from any electrically conductive material such as stainless steel, copper, Elgiloy™, MP35N, platinum, titanium, Nitinol and various other materials and alloys.

Referring to FIG. 4B, a close up view of the distal end of the catheter shows the electrode 18 with conductor wire 19 attached to the electrode. Conductor wire is located within the catheter body 17 and extends the length to the proximal end of the catheter body and is attached to the electrical connector 20. External energy source (e.g. control box 21) is coupled to the electrical connector by control cord 22 and is also coupled to dispersive electrode pad 23 in a monopolar system. FIG. 4a also shows a tissue sensor element 24 located at the distal end of the catheter. Tissue sensor element can be used to directly detect targeted tissue with well-known technologies such as impedance tissue measurement and temperature measurements. For example element may be designed as an electromyogram (EMG) element that measures the electrical activity of the ganglia and nerves. Tissue sensor element can also be a thermocouple or thermistor and used to monitor and/or control the delivery of RF energy by measuring temperature of the electrode or targeted tissue during activation. In use, electrode pad 23 is attached to the patient's skin and electrode 18 is adjacent targeted tissue (aorticorenal ganglion) creating a closed electrical circuit. When activated, radiofrequency energy travels through targeted tissue resulting in tissue ablation and modification of the aorticorenal ganglion. Aorticorenal ganglion modifying catheter 16a may also be designed as an RF bipolar device by placement of more than one isolated (not in series) electrodes 18 located approximately at the distal end of the catheter. A closed electrical circuit occurs between electrosurgical generator 21 and electrodes 18 when in tissue contact (electrode pad is not required).

Radiofrequency parameters for tissue modification include frequencies between 10 to 800 kHz with a range of 450 to 500 kHz preferred and power between 0.1 to 100 watts with a range of 2 to 10 watts preferred. Applied power control can be achieved by adjusting voltage applied to the RF tissue modifying element (power control), or by adjusting power depending upon tissue impedance measured by the tissue modifying element (impedance control) or by adjusting the power to keep the tissue modifying element containing a thermocouple or thermistor at a defined target value (temperature control). Temperature control can be in the range of 40-100° C. for a period of 5 seconds to 5 minutes. Preferably, the temperature range is 60-80° C. for a period of 60 to 90 seconds. Temperature control mechanism may also utilize a control feedback mechanism such as a proportional-integral-derivative (PID) controller or combination thereof (e.g. PI, PD controllers) to maintain target temperatures during power delivery. Aorticorenal ganglion modifying catheter employing ultrasound energy for tissue modification may utilize a piezo-electric crystal as the tissue modifying element and be coupled to external energy source as previously described. Ultrasound energy in the range of 10 KHz to 4 MHz may be applied to affect tissue modification. Aorticorenal ganglion modifying catheter may also utilize microwave energy which employs electromagnetic waves in the microwave spectrum (300 MHz to 300 GHz) for tissue modification.

Percutaneous placement of the aorticorenal ganglion modifying catheter in proximity to the aorticorenal ganglion may be accomplished using any of the currently available techniques and ancillary equipment for vascular interventions including guided sheaths, steerable distal tip assemblies and over the wire configurations employed for diagnostic and therapeutic devices. FIG. 5 is a close up view of the monopolar radiofrequency aorticorenal ganglion modifying catheter 16b placed within a guide sheath 25 and positioned within the renal artery 9 so that tissue modifying element 18 is adjacent the aorticorenal ganglion 13.

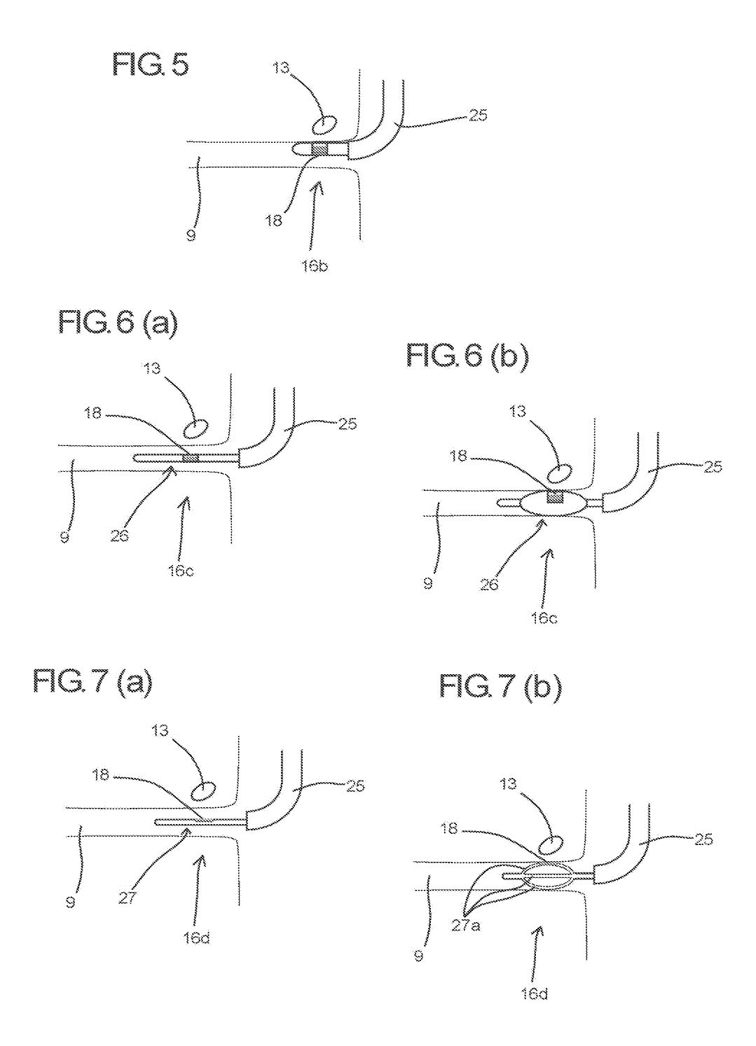

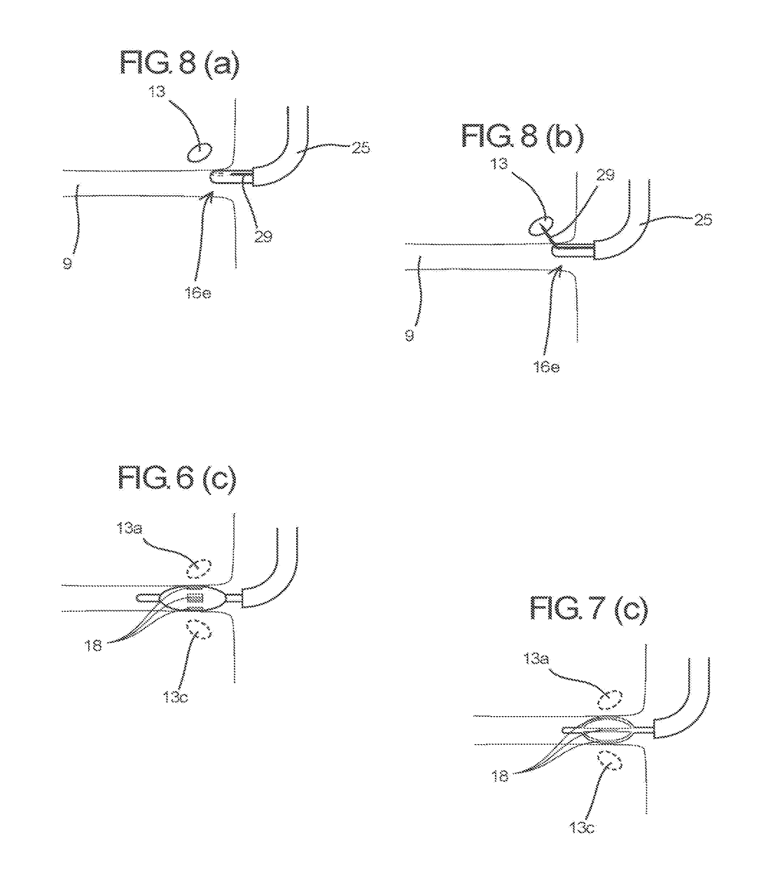

FIG. 6 is an illustration of the distal end of an aorticorenal ganglion modifying catheter assembly 16c comprising balloon element assembly 26 and tissue modifying element 18 attached to outer surface of balloon element assembly positioned within the renal artery 9. Balloon element assembly 26 is similar in design to the balloons manufactured for coronary angioplasty catheters. Balloon element may be manufactured with a relatively thin walled compliant or noncompliant plastic. Examples of materials used to manufacture the balloon element include polyethylene, polyethylene terephthalate, nylon and silicone elastomers. Balloon element assembly 26 is attached to an inflation tube (not shown) which extends longitudinally between proximal end and distal end of the catheter body 17. Balloon element assembly 26 is movable between a collapsed configuration and an expanded configuration, as shown in FIG. 6b. Balloon element assembly 26 may be inflated and deflated similarly to techniques used for angioplasty, for example by use of a pneumatic indeflator attached to the proximal end of inflation tube. In use, balloon element assembly 26 is placed at targeted treatment site within vessel lumen and inflated until electrode element 18 is contacting the vessel wall adjacent the aorticorenal ganglion 13. Tissue modification with balloon aorticorenal ganglion modifying catheter is performed similarly as described with monopolar aorticorenal ganglion modifying catheter.

A similar device 16d to the catheter assembly 16c of FIG. 6 is illustrated in FIG. 7a-c. Balloon element assembly 26 is replaced with a basket or malecot element assembly 27. Basket element assembly comprises thin rib members 27a of solid deformable material and tissue modifying element 18 attached to the outer surface of ribbon. Basket element assembly 27 is movable between a collapsed arrangement (FIG. 7a) and an expanded arrangement (FIG. 7b) with the intermediate segments of the ribbons 28 in the expanded arrangement moving laterally outward relative to the distal and proximal ends of the ribbons 28 with respect to the collapsed arrangement of FIG. 7a. Basket element assembly 27 can be expanded or collapsed by various means. One example involves manufacturing ribbons with a memory metallic alloy (e.g. Nitinol) which have a preformed expanded shape that is constrained in a catheter lumen and then allowed to recover to preformed shape upon exit of the catheter lumen. Another example involves mechanical expansion employing pull wire. Pull wire (not shown) is an elongated body extending longitudinally between a proximal end and a distal end, slidably contained within catheter body. Distal end of pull wire is attached to distal ribbon 28 ends and proximal ribbon ends are fixed to the catheter body. Basket element assembly 27 expansion occurs when pull wire is moved in a proximal and longitudinal direction relative to the catheter body causing proximal ribbon ends and distal ribbon ends to converge resulting in radially outward expansion of intermediate portion of ribbons 28.

In use, aorticorenal ganglion modifying catheter 16d containing basket element assembly 27 is inserted into targeted treatment site within vessel lumen 9 in the collapsed arrangement (FIG. 7a). Basket element assembly 27 is expanded and ceases expansion once significant resistance occurs between intermediate ribbon segments 28 and the inner vessel lumen surface (FIG. 7b). Tissue modification with basket aorticorenal ganglion modifying catheter is performed similarly as described with monopolar aorticorenal ganglion modifying catheter.

FIGS. 8a and 8b are an illustration of the distal end of an aorticorenal ganglion modifying catheter assembly 16e comprising a tissue modifying element in the form of a needle electrode 29 positioned within the renal artery 9. The needle electrode element 29 is a typically rigid or semi-ride longitudinal cylindrical structure slidably contained within catheter body comprising a sharp pointed distal end to aid with insertion into vessel wall and proximal end coupled electrically to electrical connector and attached mechanically to needle advancing mechanism (not shown). Needle electrode element 29 can be advanced or retracted by the operator by various means including wires, hand held mechanisms and handles with activation mechanism.

In use, aorticorenal ganglion modifying catheter 16 containing needle electrode element 29 is inserted into targeted treatment site within vessel lumen 9 with needle electrode element retracted within catheter body (FIG. 8a). Needle electrode element 29 is advanced from distal end of catheter and pierced and inserted into the vessel wall in proximity to the aorticorenal ganglion 13 (FIG. 8b). Tissue modification with electrode needle aorticorenal ganglion modifying catheter is performed similarly as described with monopolar aorticorenal ganglion modifying catheter. It may be desirable to control the insertion depth of the needles to accurately target the renal nerves and prevent any undesired damage to deeper tissues. Various techniques and mechanisms can be employed to control the insertion depth of the needle into the vessel wall such as adding mechanical stoppers to the needle electrode element 29. Needle element can also be designed as a hypodermic needle so that pharmacological, chemical, sclerosing, radiopaque markers, anesthetics and fluids can be delivered to tissue approximate the aorticorenal ganglion 13. Needle electrode element can also contain tissue sensor elements 24 to assist in monitoring and controlling energy delivery as well as direct detection of the aorticorenal ganglion 13 (e.g. impedance tissue measurements).

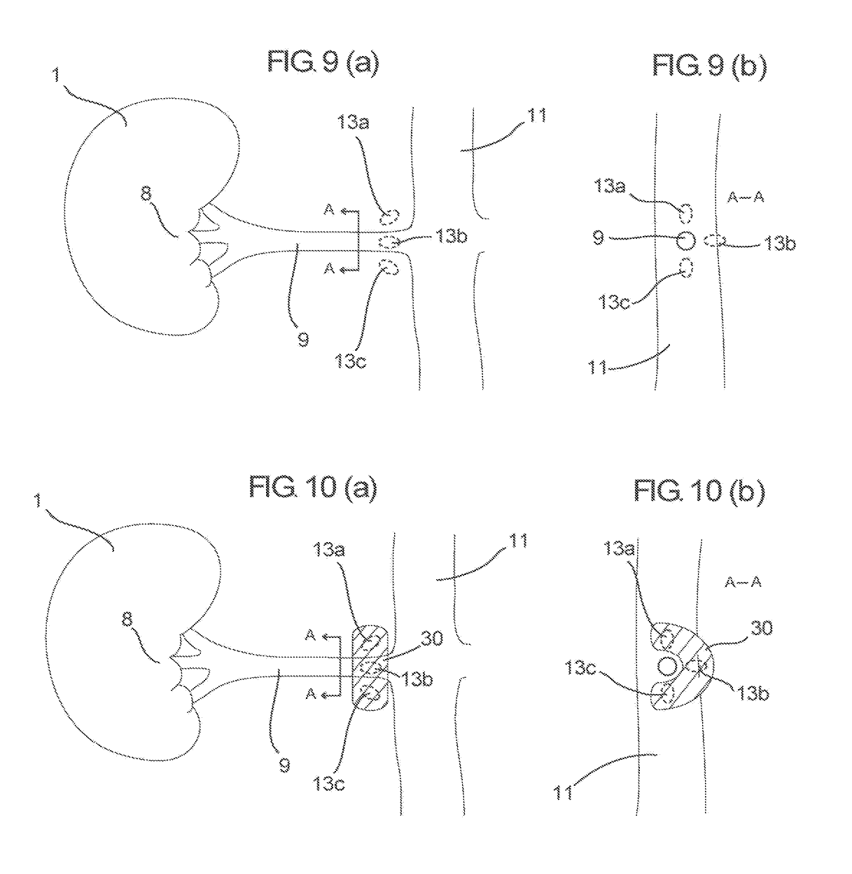

Typically, there is one aorticorenal ganglion 13 associated with each kidney 1 and is either located superior 13a, anterior 13b or inferior 13c to renal artery 9 as shown in the anterior view of FIG. 9a and sagittal view in FIG. 9b. One method of treatment involves creating tissue modification (e.g. tissue ablation when radiofrequency energy is employed) in the anatomic regions containing the aorticorenal ganglion 13. FIGS. 10a and 10b show a tissue modification zone 30 in the shape of a half toroid or half doughnut. Lesion shape can be contiguous or contain discrete segments that generally look similar to a half toroid.

Half toroid shaped lesions can be created with previously disclosed embodiments of the current invention. One method involves percutaneous placement and treatment with the monopolar radiofrequency aorticorenal ganglion modifying catheter in discrete segments along the vessel. For example, radiofrequency electrode element can be repositioned for tissue contact and activated in a superior, anterior and inferior position with the renal artery adjacent the aorticorenal ganglion. Shape of tissue modification (e.g. lesion) will generally look similar to a half toroid.

Half toroid shaped lesions can also be created with various design modifications of the previously disclosed embodiments. FIGS. 6c and 7c show the balloon and basket aorticorenal ganglion modifying catheter 16 respectively, with multiple electrode elements 18. Electrode elements are positioned in a superior, anterior and inferior configuration to create a half toroid shaped lesion capturing the aorticorenal ganglion when activated.

Well known radiographic technologies may be utilized to locate aorticorenal ganglia for treatment including intravascular and external ultrasound, magnetic resonance imaging (MRI), electromyography (EMG), nerve conduction velocity testing (NCV), somatosensory evoked potential (SSEP) and x-ray computed tomography (CT scan) and may be incorporated into the aorticorenal ganglion modifying catheter 16

Aorticorenal ganglion and/or renal nerves (e.g., the postganglionic nerves 14 located between the ganglion 13 and the kidney 1) can be detected by stimulation with a tissue stimulating element and measurement of a physiological response with a physiological measurement element. Tissue stimulating element and/or physiological measurement element can be separate catheters or incorporated as elements into aorticorenal ganglion modifying catheter. Physiological measurement element (sensor) located approximately at the distal end of the catheter may function by transmitting data collected at the sensor to an external system for analysis. Transmission of data may be accomplished by various means including delivering a signal from sensor through a signal transmitting conduit located within the catheter body that extends the length to the proximal end of the catheter body. Proximal end of catheter body is coupled by way of connectors and/or cables to system, to a software containing system for analysis. For example a pressure sensor, such as a pressure transducer, located at the distal end of the catheter transmits electrical data from sensor through the catheter body to system for analysis.

Physiological data may be analyzed by software for determining ganglion location and treatment verification by various means. One method of determining ganglion location involves comparing non-stimulated tissue physiological data to stimulated tissue physiological data with pre-set limits established to ascertain positive and negative results for ganglion detection. For example, when intravascular Doppler ultrasound is utilized for physiological response, blood flow velocity measured as centimeters per second would decrease significantly when ganglion is stimulated due to renal vasoconstriction compared non ganglionic tissue stimulation.

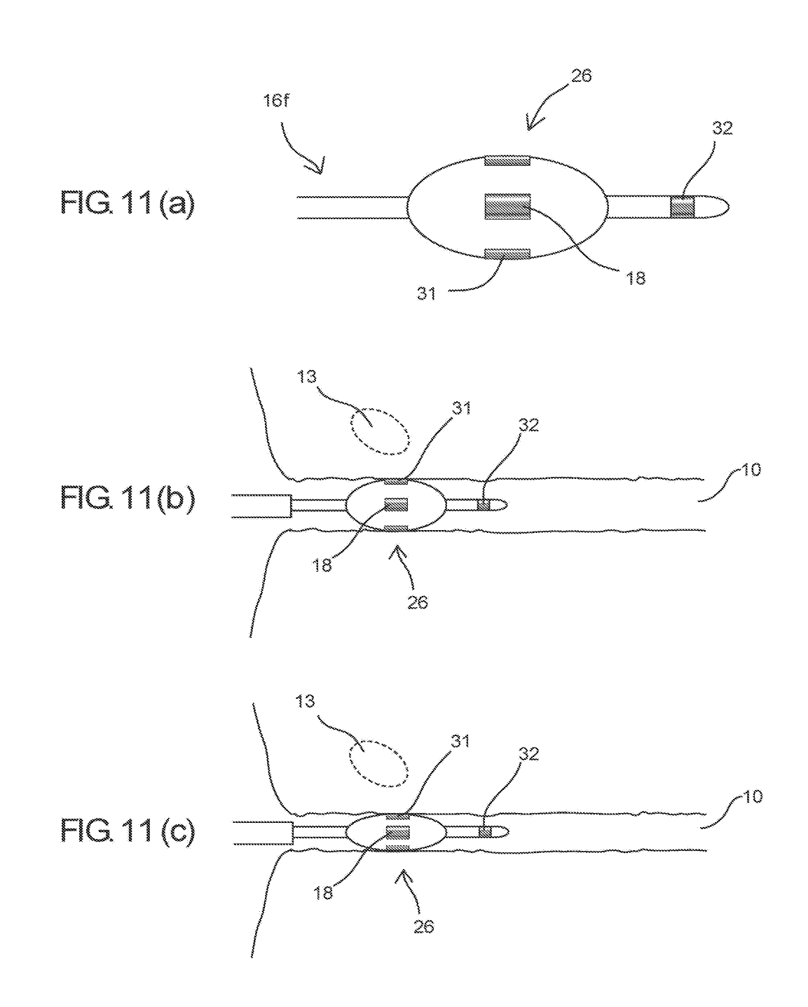

FIG. 11(a) is a close up view of the distal end of an aorticorenal ganglion modifying catheter 16f that is capable of stimulation, sensing, and modifying tissue. The catheter comprises a balloon element assembly 26 having a tissue stimulating element 31, a sensing or physiological measurement element 32, and a tissue modifying element 18.

The tissue stimulating element 31 which utilizes electrical current for operation can be manufactured from any electrically conductive material such as stainless steel, copper, Elgiloy™, MP35N, platinum, titanium, Nitinol and various other materials, and alloys. Similar materials may also be used as tissue modifying element 18. The physiological measurement element 32 comprises a sensor or sensors such as a pressure transducer, ultrasound transducer, optical coherence tomography sensor, temperature sensors and the like. The tissue modifying element, tissue stimulating element and physiological measurement element may also be comprised of a nanoelectronic, flexible electronic, flexible sensor, microsensor, stretchable electronic and the like. FIG. 11(b) is an illustration of the distal end of the aforementioned catheter positioned within the renal artery 10 before the application of electrical stimulation. Preferably, the catheter 16f is connected to and operated via a control box 21, as described in detail elsewhere in this specification.

FIG. 11(c) is an illustration of the physiological response of renal vasoconstriction during stimulation with tissue stimulating element 31. The reduction in inner luminal diameter of the vessel is detected by sensor 32 (e.g. measurement of diameter change with ultrasound transducer or blood pressure measurement) and/or is detected under fluoroscopy by observing radially converging radiopaque tissue stimulating elements 31.

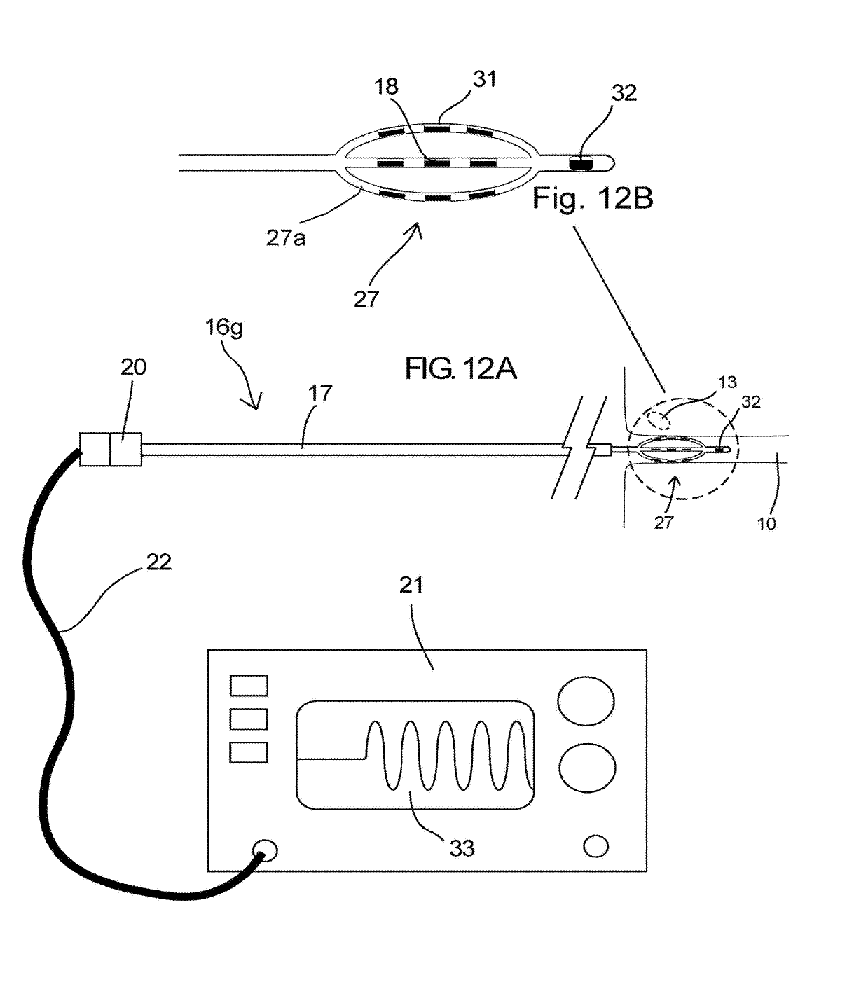

FIGS. 12a and 12b are schematic views of an aorticorenal ganglion modifying catheter 16g that is capable of stimulation, sensing, and modifying tissue. Specifically, the catheter 16g comprises a distal basket element assembly 27 including tissue modifying elements 18, tissue stimulating elements 31, and sensor 32. The basket element assembly 27 can be formed from a plurality of rib member 27a (e.g., between 3 and 10 rib members 27a) and can be configured to radially expand from a compressed configuration via a manual expansion mechanism (e.g., a control wire) or via self-expansion (e.g., superelastic shape memory material).

Each rib 27a of the basket 27 can include at least one stimulating element 31 and one modifying element 18, and more preferably, several of each elements on each rib. Preferably, the sensor 32 is located distally and spaced apart from the elements 18 and 31, however, in an alternate embodiment, one or more sensors 32 can also be located on the ribs of the basket 27. It should also be understood that while a basket 27 is described, any number of shapes and materials can be used, such as a spiral or coil shape, a tubular shape, or a balloon.

While it is contemplated that all of the stimulating elements 31 can be activated in unison and all of the modifying elements 18 can be activated in unison, less than all of each group of elements can also be activated to allow the location of the aorticorenal ganglion to be better targeted (e.g., radially and axially). For example, the control box 21 (either manually or automatically) may initially only activate the stimulating elements 31 on one or two ribs of the basket 27 at a time, allowing the user or software in the control box 21 to determine the rib 27a closest to the aorticorenal ganglion 13. In another example, the software in the control box 21 may activate and deactivate the stimulating elements 31 in a predetermined pattern, such as consecutive, adjacent ribs. In another example, the user or software in the control box 21 can activate all of the proximal, distal, of middle sensors of each group, allowing the user or control box 21 to determine if the aorticorenal ganglion is located proximally, distally, or immediately adjacent to the basket 27. In yet another example, any combination of the above described element activation can be used (e.g., only the distal stimulating elements 31 on a single rib 27a of the basket 27 can be activated.

The control box 21 preferably includes controls and a visual display 33 to provide information to the user, such as a measured physiological response (e.g., blood pressure data from sensor 32) or status of any of the elements (e.g., whether the tissue modification element 18 is turned on). In one embodiment, the visual display 33 is a touch screen. Additionally, the control box 21 includes software configured to operate the components on the catheter 16g, display simple data points or stream real time data, and also provided visual and audible procedure instructions during operation. The control box software may also control the catheter to prevent certain undesired modes of operation, and to control operation of the catheter in the event of an interruption in proper operation. While the control box 21 is depicted as a separate, standalone unit, it is also contemplated that it could be incorporated into the handle or proximal end of the any of the catheters described in this specification.

In operation, the distal end of catheter 16g is positioned within the renal artery 10 (or alternately in the renal vein 5) and proximal end of the catheter 16g is connected to the control box 21 via the control cord 22. Next, the user interfaces with the control box 21 to begin a stimulation and sensing routine. As previously discussed, such a routine may include sensing with the sensing element 32 while all of the stimulating elements 31 are activated or while only select portions are activated (e.g., elements 31 on only a single rib 27a and/or in the proximal, middle, or distal portions).

Once the sensor element 32 and control box 21 detect and display the appropriate change in physiological data (e.g., blood pressure pulsation), the modification elements 18 (or a portion thereof), are activated. This activation can be manually activated on the control box 21 by the user or automatically performed by the software in the control box 21 based on the data from the sensing elements 32.

Finally, the stimulating elements 31 and sensing element 32 are again activated (or optionally are continually activated during the entire process) to allow confirmation that the aorticorenal ganglion (or possibly another renal nerve location) has been treated to adequately limit or prevent nerve signals from reaching the kidney. Again, this confirmation may be performed manually by the user by looking at data on the visual display 33 or automatically by the software of the control box 21 (which may further indicate confirmation via an audible and/or visual signal). While this process of use was described in connection with catheter 16g, it should be understood that any of the other embodiments described in this specification can be used in a similar fashion (e.g., alone for a catheter having stimulating, sensing and modification elements, or several different catheters that each contain one or more of these elements).

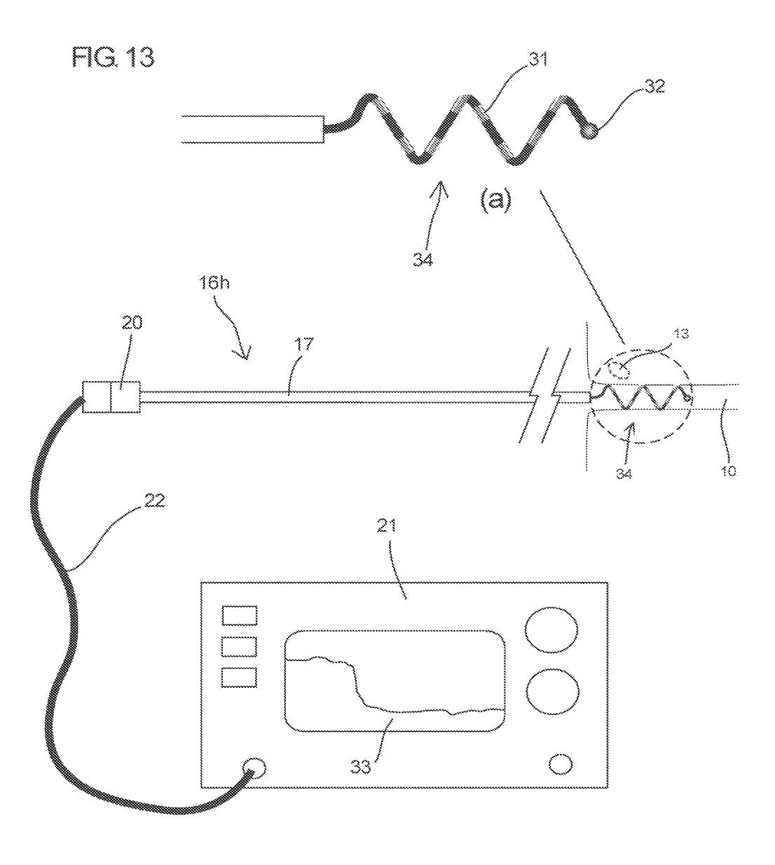

FIG. 13 is a schematic of the aorticorenal ganglion modifying catheter 16h comprising a distal coil element assembly 34 comprising tissue stimulating elements 31 and sensor 32. Distal end of catheter 16h is positioned within the renal artery 10 and proximal end of the catheter is connected to a control box 21 comprising a visual display 33. In use, visual display shows the operator a detected response, for example during stimulation of the aorticorenal ganglion with stimulating element 31, blood flow velocity can be detected with sensor 32 comprising a Doppler ultrasound transducer and exhibited on control box visual display 33.

Aorticorenal modifying catheter may also comprise a lumen within the catheter body that extends from distal end to proximal end of the catheter body. Catheter lumen allows for slidable placement of a guide wire which is used to assist with placement in the renal vasculature as commonly performed for percutaneous procedures utilizing a guide wire. Catheter lumen may be designed for rapid exchange of multiple catheters with a stationary guide wire by various means for example by comprising a radial slit from the lumen to the outside surface of the catheter body that extends longitudinally approximately half the length of the catheter. Lumen may also be used for placement of tissue stimulating element and physiological measurement element (e.g. FloWire® Doppler Guide Wire and Verrata™ Pressure Guide Wire).

Stimulating element and tissue modifying element may be activated separately or simultaneously with the latter allowing for a cessation of tissue modification once acceptable nerve disruption, as measured by a physiological response, is achieved. Stimulating element utilizing radiofrequency energy may be a monopolar or bipolar arrangement, connected to an external electrical stimulator or electrosurgical generator capable of delivering adequate electrical parameters for ganglion or nerve stimulation. Nerve stimulation may be achieved with frequencies between 0.1 to 100 Hz with a range of 2 to 50 Hz preferred, voltage between 0.1 to 30 volts with a range of 5 to 15 volts preferred and pulse duration between 0.1 to 10 ms with a range of 0.2 to 5 ms preferred. One set of stimulation energy parameters or variation of parameters may be utilized for tissue stimulation. For example, lower frequencies (e.g. 2 Hz) may be used to detect efferent nerve physiological responses and higher frequencies (e.g. 50 Hz) may be used to detect afferent nerve physiological responses. Frequency modulation may occur in series, parallel, simultaneously, as a slope function or step function or any combination thereof. Voltage, current and pulse duration may also be varied during stimulation to achieve desired physiological responses of the ganglia and nerve tissue. A single control box may be used for tissue stimulation, physiological response analysis and tissue modification.

While the stimulating elements 31 and the tissue modifying elements 18 in any of the embodiments of this specification may be separate, dedicated electrodes (i.e., only used for one purpose), it is also contemplated that each electrode can operate as either type of electrode. For example, the electrodes may be connected to a current-generating source in the control box 21 that is capable of producing aorticorenal ganglion stimulating current and tissue modifying current (as described elsewhere in this specification).

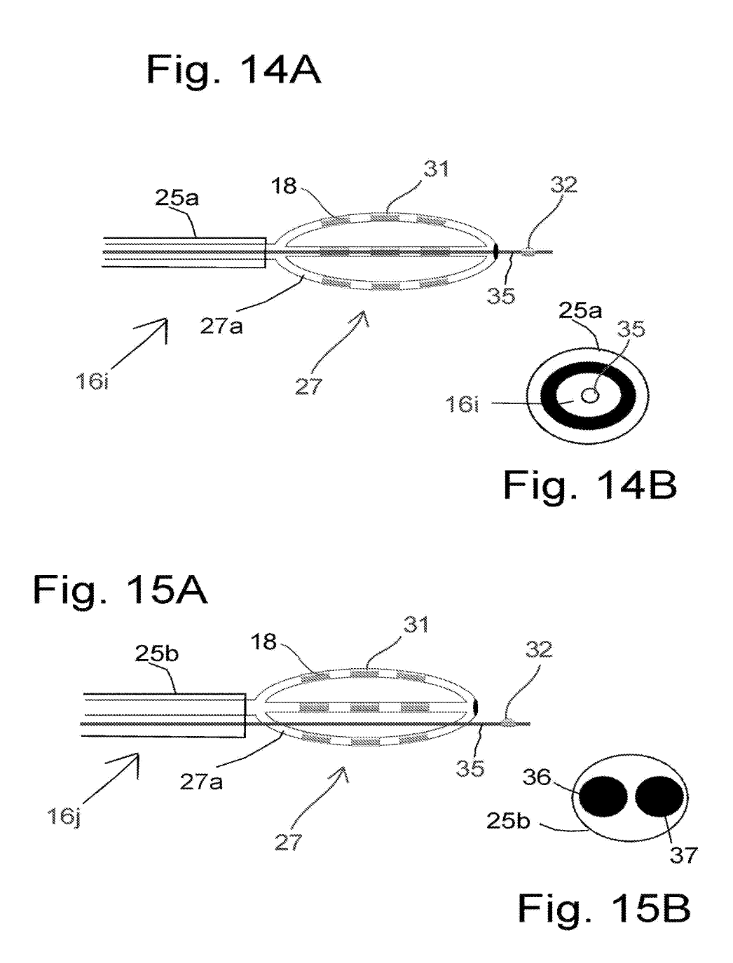

Turning to FIGS. 14A and 14B, an aorticorenal ganglion modifying catheter assembly 16i is illustrated within a single lumen guide sheath 25a. The catheter 16i also includes an interior lumen that opens at the distal end of the basket 27 and the proximal end of the catheter 16i, allowing a separate sensor catheter 35 with sensing element 32 to be separately moved relative to the basket portion 27a. In another embodiment FIGS. 15A and 15B illustrate an aorticorenal ganglion modifying catheter assembly 16J located within a first lumen 36 of a guide sheath 25b and a separate sensor catheter 35 located within a second lumen 37.

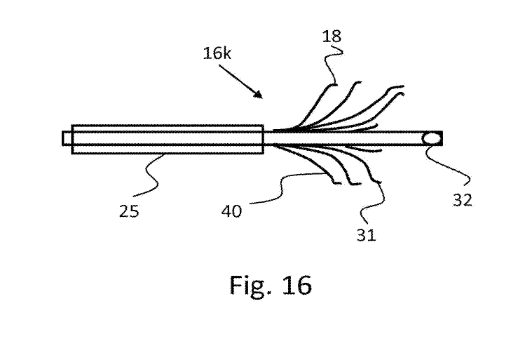

FIG. 16 illustrates another embodiment of an aorticorenal ganglion modifying catheter assembly 16k which is generally similar to the previously described embodiments, but includes a plurality of paddles or arms 40 having on or more of the stimulating elements 31 or the tissue modifying elements 18. Preferably, the arms 40 are composed of superelastic material (e.g., Nitinol) and configured or biased to self-expand radially outward from the main body. In one example, the entire arm 40 includes a conducting material, allowing the entire arm 40 to act as either the stimulating element 31 or the tissue modifying element 18. In another example, the arms 40 may each include a wire or similar conductive path which connects to the stimulating element 31 or the tissue modifying element 18 at its tip.

There are other methods not employing electric current to stimulate ganglia or nerve tissue such as of a chemical or drug that stimulates the targeted tissue. For example, adrenergic drugs stimulate sympathetic nerves by either mimicking the action of the neurotransmitter norepinephrine or stimulating its release. Examples of adrenergic drugs include epinephrine, norepinephrine, isoproterenol, dopamine, dobutamine, phenylpropanolamine, isoetharine, albuterol, terbutaline, ephedrine and xylazine. Drugs can be delivered by various means including by use of previously described hypodermic needle electrode element 29.

### Experiment 1

Chronic swine study was performed to demonstrate reduction in renal nerve activity after modification of the aorticorenal ganglia. The domestic swine model is an established model for the renal system because the pig renal anatomy, including circulatory and nervous system, is similar to that of humans.

The procedure involved placing the anesthetized test animal in dorsal recumbency, followed by a 10-cm midline abdominal incision in order to access the renal anatomy. Peritoneum was removed to expose left and right renal artery, vein, aorta and vena cava. Adventitia was stripped from the renal arteries and veins to expose the renal nerve plexus and aorticorenal ganglia. Direct electrical stimulation of the ganglia was performed at 15 volts, 5 Hz and 0.5 msec. pulse duration using a Grass Instruments SD9 Square Pulse Stimulator (Grass Technologies, Warwick, R.I.). Proper identification of the aorticorenal ganglion was confirmed during stimulation by observation of renal artery constriction and kidney blanching (renal vasoconstriction). Aorticorenal ganglia were surgically removed bilaterally and captured for histopathology and the abdomen sutured in two layers at the conclusion of the surgical excision procedure.

At approximately 7 days, the animals were sacrificed and renal cortical samples were removed for measurement of renal cortex norepinephrine levels. Norepinephrine is a neurotransmitter secreted at the end of nerves and is measured to determine nerve activity and is a surrogate for measuring renal denervation success in animals. Two test animals with histologically confirmed aorticorenal ganglia removal were compared to 2 naive control animals. Renal norepinephrine was reduced 72% in the test animals compared to the controls.

### Experiment 2

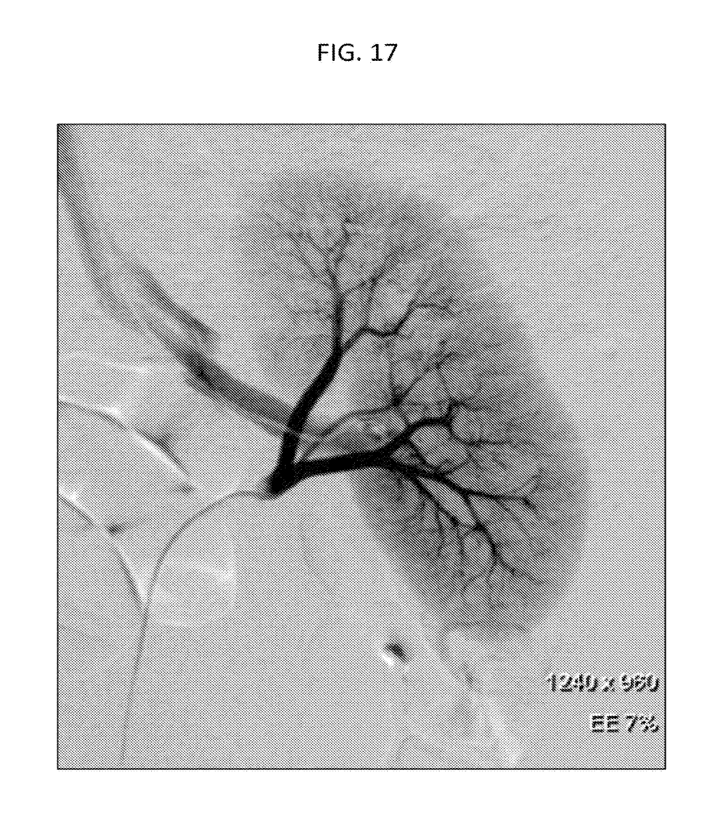

An acute swine study was performed to evaluate the feasibility of detecting an acute physiological response to percutaneous stimulation of the aorticorenal ganglion and renal nerve tissue. The procedure involved creating percutaneous access to the renal venous and arterial vasculature through a jugular and femoral puncture site of an anesthetized test animal in dorsal recumbency. Guide sheaths used for radiopaque contrast delivery were placed with fluoroscopic guidance in both the left renal vein and left renal artery to perform a baseline nephrogram. FIG. 17 is a frame capture of the baseline nephrogram showing normal intrarenal vessel emptying and kidney perfusion.

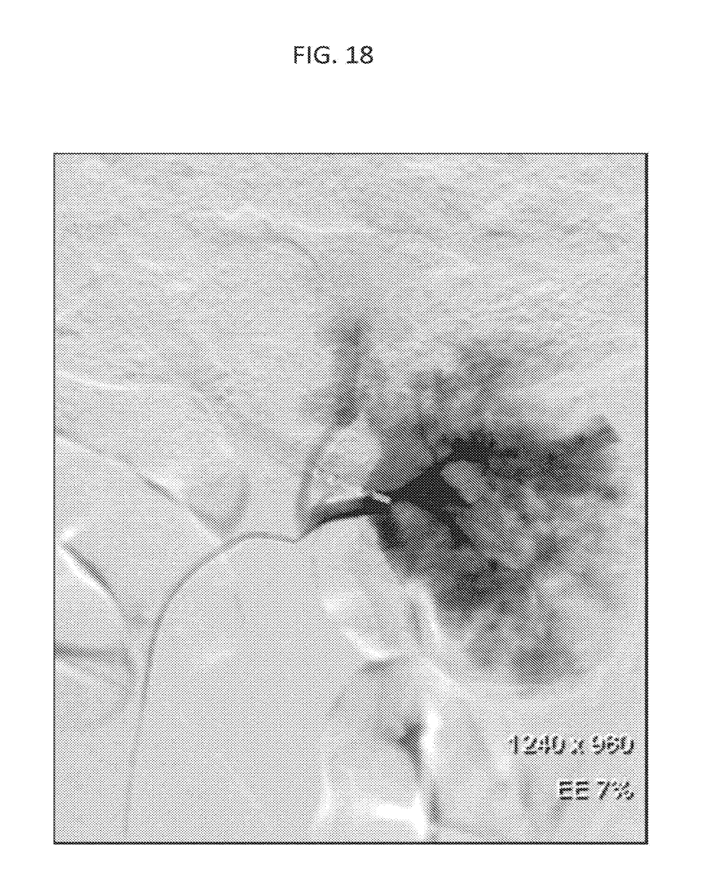

A modified electrophysiology catheter (5 French Marinr™ Ablation Catheter, Medtronic, Minneapolis, Minn.) attached to a Grass Instruments SD9 Square Pulse Stimulator (Grass Technologies, Warwick, R.I.) was percutaneously placed in the left renal vein. Direct stimulation of the renal vein wall was performed at 15 volts, 5 Hz and 0.5 msec. pulse duration at several locations simultaneously with contrast delivery to the left renal artery to observe physiological responses. FIG. 18 is a frame capture of a nephrogram performed with stimulation demonstrating activation of the efferent renal sympathetic nerves resulting in renal vasoconstriction and decreased renal blood flow.

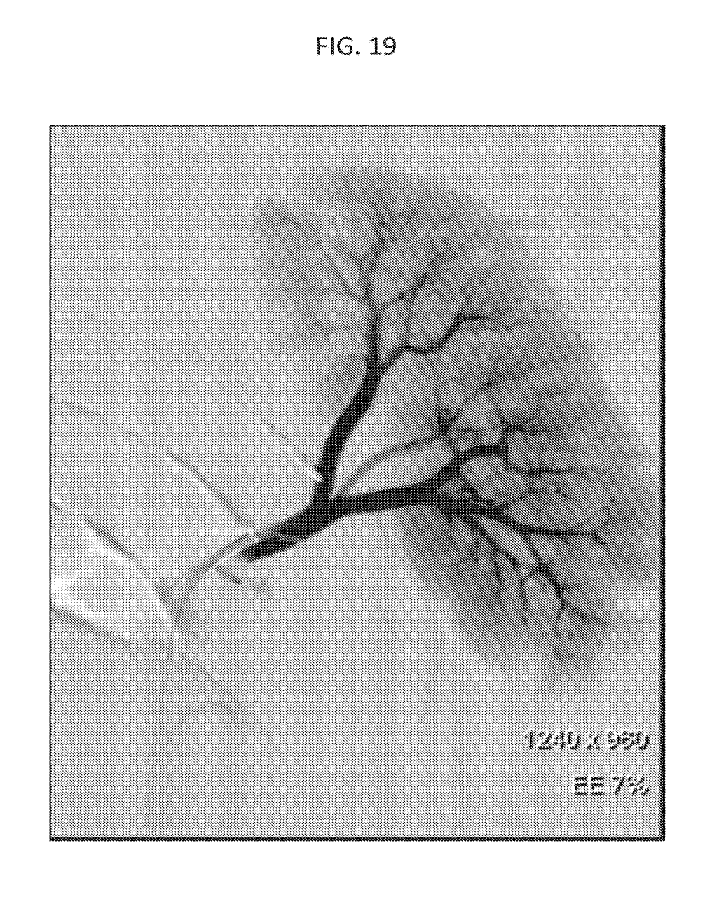

After stimulation, catheter was disconnected from stimulator and connected to an electrosurgical generator (Radionics RFG3, Burlington, Mass.). Radiofrequency energy was delivered at an electrode temperature of 70° C. for a period of 90 seconds to ablate the adjacent tissue. Following RF energy delivery, catheter was reconnected to the stimulator and repeat stimulation performed. FIG. 19 is a frame capture of the nephrogram during repeat stimulation showing similar intrarenal vessel emptying and kidney perfusion compared to baseline thus indicating disruption of the renal nerve path. These results demonstrate that a renal physiological response can be detected with percutaneous stimulation and also verification of ganglia or nerve tissue wounding can be determined by reapplying stimulation and analyzing the resulting physiological responses.

There may be other means to modify the aorticorenal ganglia not specifically described in one of the inventions embodiments, but it is to be understood that the description is not meant as a limitation since further modifications may suggest themselves or be apparent to those skilled in the art.

While the present specification has primarily described the detecting and treatment of an aorticorenal ganglion, it should be understood that the same devices and methods can be similarly used to detect and treat any portion of the renal nerves between the aorticorenal ganglion and the kidney.

The following portion of the specification generally contains 5 sections as follows:
1. Discovery Section: This section describes methods, apparatuses, and usage cases for discovering the Target Location of the ARG.
2. Treatment Section: This section describes methods, apparatuses, and usage cases for treating the ARG
3. Confirmation Section: This section describes methods, apparatuses, and usage cases for determining if the aforementioned treatment is successful.
4. Mesh Catheter Section: This section describes a novel aorticorenal ganglion modifying catheter.
5. Experiment 3 Section: This section describes a chronic animal study using some of the techniques disclosed in the discovery and treatment section.

This section of the disclosure describes methods and apparatuses used to determine the Target Location, i.e. a location in close proximity to the ARG. In order to apply a treatment procedure to the ARG it may be first necessary to determine the Target Location. The procedure used for finding the Target Location of the ARG is referred to as the “discovery” procedure. The Target Location can be used to determine a Target Volume, where the Target Volume is defined to be a volume that encompasses the aorticorenal ganglion.

The Following are Examples of Methods that May be Employed to Determine the Target Location:

Method 1:

This discovery procedure uses electrical stimulation and measures change in monitored patient physiological parameters during and after stimulation.

Note that for this method, it may be necessary to first take measurements of the monitored parameter(s) prior to any stimulation to establish a baseline behavior. After the stimulation has been performed the same measurement protocol is repeated and the results are compared with the baseline results. The magnitude of the response (to simulation) is the difference between value of the parameter monitored during and after stimulation and the value of the parameter measured before stimulation has been applied (the baseline value). Therefore the response is a function of time. Various functions of the response versus time may be used to characterize the response over time, e.g. root mean square value of the response over a given time segment after initiation of stimulation.

Consider a discovery procedure that uses an electrode catheter in the renal artery to apply electrical stimulation. Electrical stimulation can be used to determine the location within the lumen of the renal artery that is closes proximity to the ARG, i.e. the “Target Location”. Application of treatment at the Target Location, or in the Target Volume, has the advantage of achieving successful treatment with the least amount of RF energy, thereby minimizing collateral damage. It has been shown in Experiment 3 that a stimulation voltage applied to an electrode carried by a catheter placed in direct contact with the superior inner wall of the renal artery within an effective range of the ARG will elicit a change in the baseline state of one or more parameters, e.g. renal artery blood flow velocity, renal artery blood flow, renal artery blood pressure, renal artery diameter, etc. In addition Gal, and company (P. Gal, et al, Journal of Hypertension 2014: 1-4) found that stimulation also induced systemic changes, i.e. changes in aortic blood pressure and heart rate. One can also monitor electrical activity of electrical signals propagating down the nerves that innervate the renal artery, also known as renal sympathetic nerve activity (RSNA).

As mentioned earlier, to measure changes in the monitored parameters it is first necessary to establish the baseline behavior/state. Once this is established, a search protocol is initiated that determines the magnitude of the response as a function of stimulating electrode position, which can be accomplished by either physically moving the catheter, or in the case of a multi-electrode catheter selecting which electrode(s) are connected to the stimulation generator. Because the ARG is generally located superior to the renal artery the electrodes chosen for the stimulation search should initially be in contact with the superior portion of the renal artery. The location of the Target Location is approximately located at the electrode position which yields the largest change behavior as a function of distance from the ostium of the renal artery. The characteristics of the stimulating waveform, e.g. wave shape, amplitude, frequency, duty cycle, etc., are chosen to be sufficient to show a definitive response and yet not so strong as to induce a spasm in the renal artery, which may delay the application of the treatment.

The nature of that stimulation may be monopolar or bipolar. The distinction between monopolar stimulation and bipolar stimulation is the proximity of return electrode position relative to the stimulating electrode position. An example of monopolar stimulation is if the return electrode is a dispersive pad or plate generally located on the external surface of the patient, approximately 10 or more centimeters from the stimulating electrode. The electric field subtended between the stimulating electrode and the return electrode induces currents outside of the area of treatment. For bipolar the location of the return electrode may be less than a few centimeters. Because the extent of the electric field is more constrained in the bipolar case the bipolar stimulation has the advantage that it may reduce the chance of inducing muscle contraction and/or stimulation of sensory nerves outside of the volume of concern, thereby reducing the risk of discomfort to the patient.

Method 2

Renal sympathetic nerve activity (RSNA) is the electrical activity of the signals carried by the ARG's post ganglionic nerves. This method uses electrical amplifying receivers coupled to selected electrode(s) to measure RSNA. Because these signals are extremely weak (even with the pre-amplification mentioned in the confirmation section of this application) the range of signal detection is limited to detecting electrical activity of signal carried by the nerves that are directly adjacent to the selected electrode(s).

As shown in FIG. 3, the highest concentration of post-ganglionic axons 14 exits the ARG 13 towards the superior surface of the renal artery 9, this location may be detected by moving the receiving electrode along the axis of the renal artery. For example, if one starts with the receiving electrode at the ostium of the renal artery and moves the receiving electrodes distally in small increments towards the renal artery bifurcation then the measured signal amplitude should markedly increase near the Target Location. Alternatively, one could start with the receiving electrode at or near the bifurcation and move medially towards the ostium. In either case the Target Location is at the location where the received signal markedly changes in amplitude or frequency. Alternatively, to physically moving the electrode(s) attached to receiving amplifier one can use a catheter with multiple electrodes and select which electrode(s) are connected to the receiver amplifier.

Method 3

Advance imaging techniques have sufficient resolution to determine the location of the ARG. For example, advanced MRI imaging techniques are capable of measuring features of 1 mm or less (Ty K. Subhawong, et al, Skeletal Radiology, 2012 January; 41(1):15-31). This method establishes the actual location of the ARG. One could use a multitude of treatment options once this location has been established. For example, if RF ablation was the preferred treatment then this method would enable the practitioner to position the RF electrode of the treatment catheter at the Target Location in the renal artery.

The actual location of the treatment depends on the type of ablation used. For the case of RF treatment, electrodes locations should be placed such that a significant portion of the current that flows between the electrode connected to one terminal of the RF electrical surgical generator and the electrode connected to the other terminal of the RF electrical surgical generator should flow through the ARG and its surrounding tissue. For example, if RF monopolar treatment is used then one terminal of the RF electrical surgical generator would be connected to an electrode at the Target Location and the other terminal of the RF electrical surgical generator would be connected to a dispersive conductive pad placed on the lower back of the patient. If bipolar is used, then the location of the source electrode depends on where the return electrode is located. One type of bipolar treatment would place the source electrode on one side of the Target Location and on the other side would be the return electrode.

### Apparatus for Implementing Aforementioned Discovery Methods

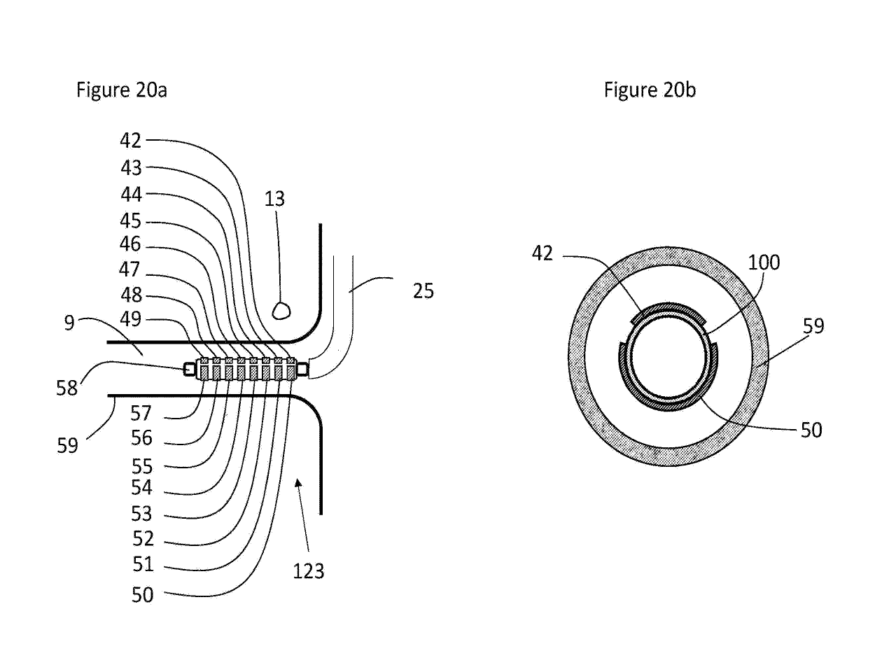

Consider a catheter shown in FIG. 20a where the distal end of the catheter has a catheter distal assembly construct in its minimal radial aspect state (i.e., a radially unexpanded state). The catheter distal assembly preferably can change its radial aspect depending on the step of the procedure, allowing it to radially expand and contract as desired by the physician.

When the catheter distal assembly is inserted into the circulatory system, a small radial size is beneficial to allow it to be inserted in a guide catheter of a nominal diameter so that it can be easily be moved through the length of the guide catheter (or guide sheath) 25 until it reaches its destination near the ostium of the renal artery lumen 9 and the approximate location of ARG 13. The catheter distal assembly houses electrode elements 42 through 57 and at least one sensor element 58. Electrode elements 42 through 49 are located on the superior side of the catheter distal assembly, located at approximately −45 degrees to +45 degrees (where 0 degrees is the superior vector angle), while electrode elements 50 through 57 approximately extend over the remainder of the angular aspect. FIG. 20b shows a cross sectional view of the device in FIG. 20a, where in this contracted state the electrode elements are not in contact with the renal artery wall 59.

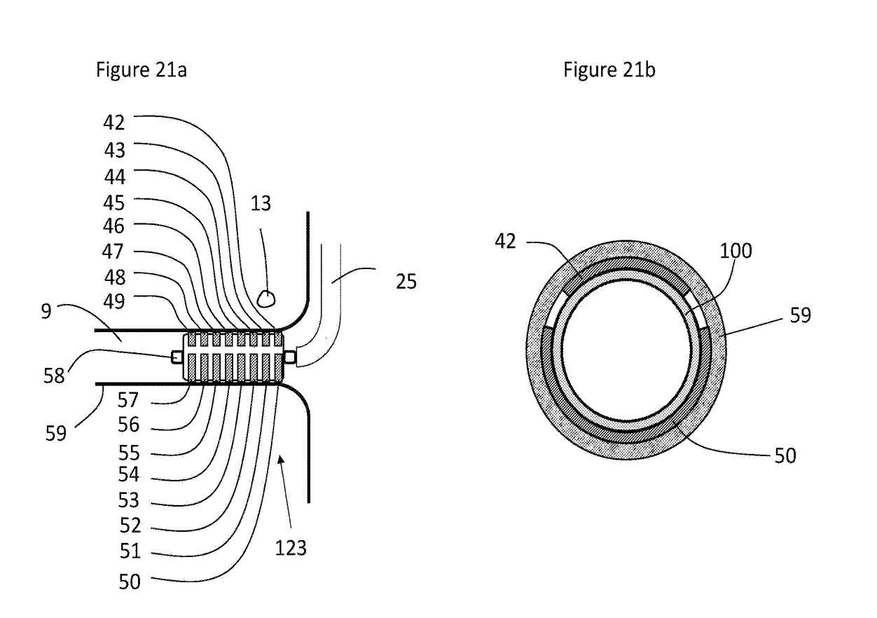

In FIGS. 21a and 21b, the catheter distal assembly has been expanded such that all of the electrode elements 42 through 57 are in contact with the renal artery wall 59.

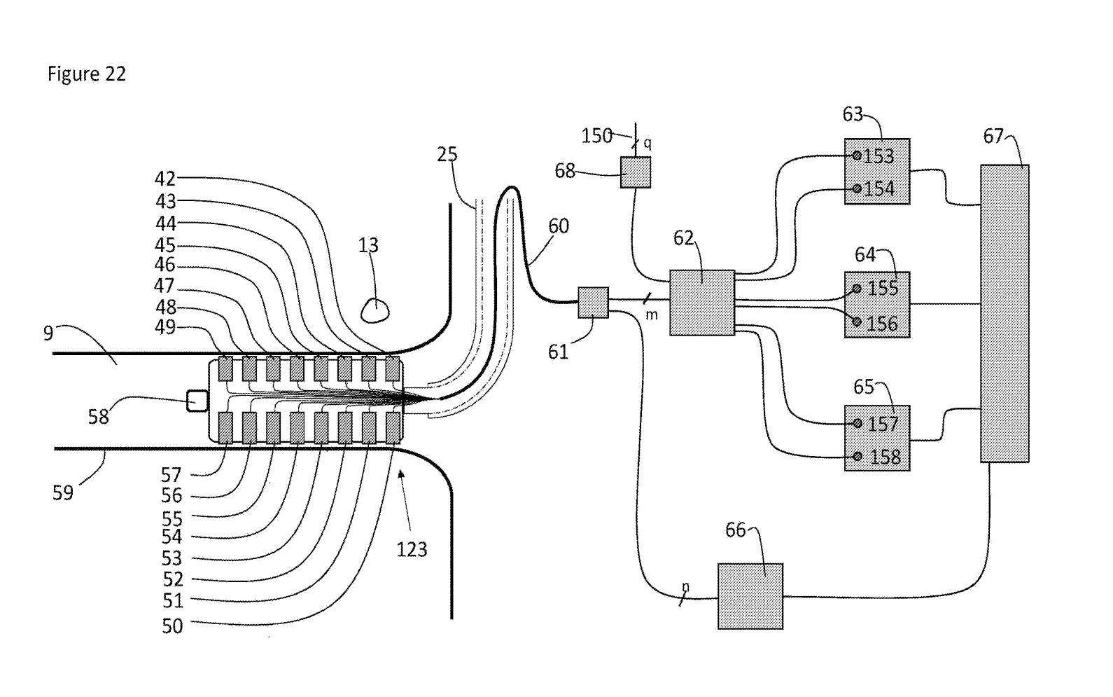

FIG. 22 shows the wiring diagram of the catheter to external functional elements 61 and 62. The bundle of wires 60 contain all the wires for each electrode element and in addition carries the wires for the sensor element 58. In other implementations that follow, the designator 60 refers to a bundle of wires, cables, and fibers that connect the catheter distal assembly with the remainder of the system. The junction box 61 breaks out the sensor element wires to sensor control and monitor unit 66. The remainder of the wires in the bundle of wires 60 are terminated at the input of the cross connect module 62. The cross connect module 62 can connect any electrode element wire with the terminals of stimulation signal generator 63, detector/receiver unit 64, or RF electrical surgical generator 65. This gives the system the ability to program any electrode element as a stimulation generator source element or return element, detector/receiver element, or RF ablation element, or a no connect state.

All of these functional elements are controlled by a central processor 67. In the case that the signal return path is not from the catheter distal assembly, for example in the case of a dispersive pad used in monopolar stimulation and ablation, there is an additional input to the cross connect module 62, from the multiplexer unit 68, which selects a return electrode from the return wire bundle 150 that is connected various return electrode options It is termed a multiplexer unit because it may receive input from not only a dispersive pad but also from other endovascular placed catheters, for example a catheter electrode that is placed in the common hepatic artery.

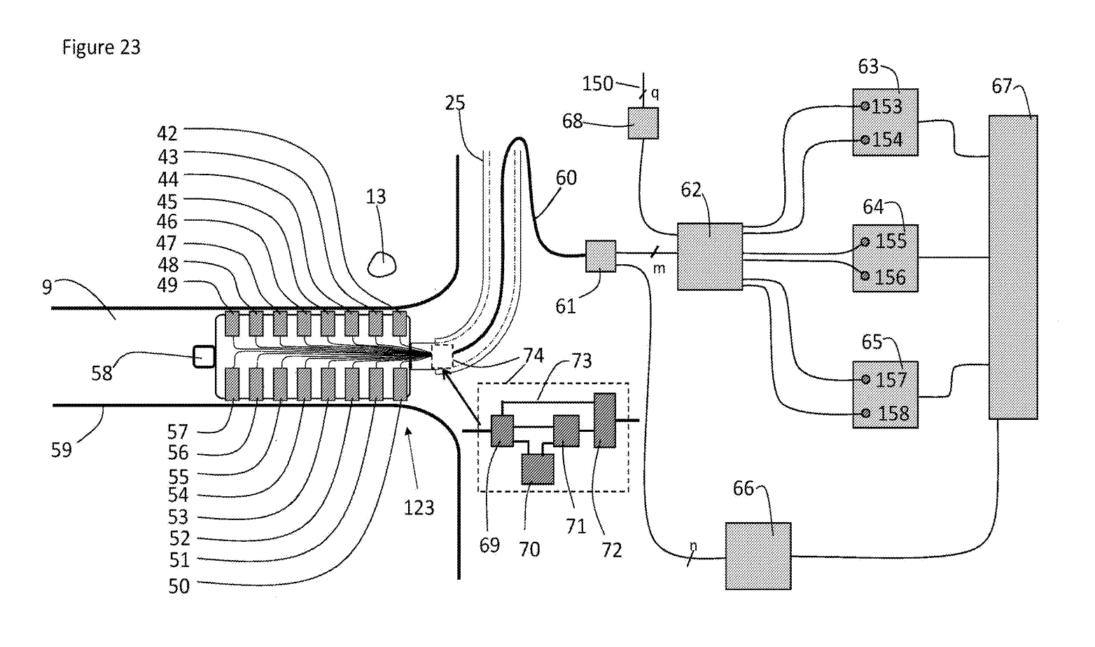

There may be certain advantages in partitioning the system functionality such that some of the functions can be implemented in the catheter distal assembly body itself. For example, in the case of the detector/receiver it may be advisable to have a preamplifier in the catheter distal assembly to reduce the effect of noise that may be introduced by extraneous sources, e.g. power lines, miscellaneous equipment electro-magnetic interference (EMI). One possible embodiment of this idea is shown in FIG. 23 where module 74 is an example of an implementation that enables preamplification in the catheter distal assembly 123. Cross connect module 69 connects and electrode element with a preamplifier module 70. The preamplifier module 70 contains one or more preamplifiers 75. This cross connect module is controlled by signal lines 73 which come from junction box 72 which parses out the upstream signals (wires from the central processor 67) from the downstream signals (wires from either wires directly connected to the electrode elements 42 through 57 or wires attached to the output of the preamplifier module 70). Junction box 71 combines those downstream signals from either the output of the preamplifier module 70 or directly from the electrode elements.

### Example of Use Cases of Apparatus for Implementing Aforementioned Discovery Methods

Case 1: Implementing Method 1 Using the Monopolar Option

Consider FIG. 22 as the schematic for an apparatus for implementing method 1 using the monopolar option. As discussed previously, the apparatus can be configured to connect any of the electrodes on the catheter distal assembly to the terminals (153 and 154) of the simulation generator 63. In this example specific electrodes on the catheter distal assembly are connected to terminal 153. Terminal 154 is connected to the dispersive pad throughout the procedure described. For this discussion, the sensor element 58 is measuring blood velocity and it is connected to monitor unit 66. The catheter distal assembly is positioned such that the electrodes 42 through 49 are in good electrical contact with the superior side of the wall of the renal artery 59.

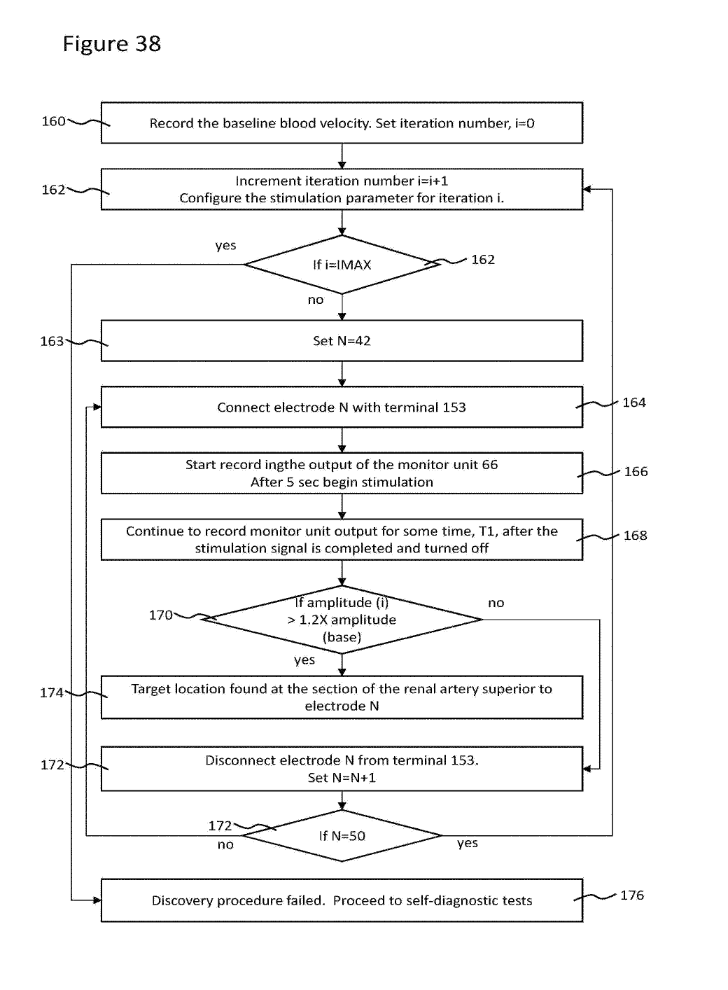

The following is an example of a search algorithm that searches for the Target Location. See FIG. 38 for a flowchart diagram of this algorithm. It starts by stimulating the catheter electrode closest to the ostium, i.e. electrode 42 and subsequently selecting the next electrode immediately distal to the one that was previously stimulated. This process continues until either a stimulation response of sufficient magnitude is detected or the last electrode 49 has been stimulated. If none of the stimulations at electrodes 42 through 49 elicit a significant response, then the stimulation parameters are adjusted to a new stimulation parameter set and the process is repeated. If a Target Location cannot be located after completing all sets of the stimulation parameters, then a diagnostic test must be run to determine the root cause of the failure of the discovery procedure.

Example of a Search Algorithm:

(step 160) Record the baseline blood velocity prior to stimulation. Set iteration number, i=0.

(step 162) Increment iteration number i=i+1. If i=IMAX then go to step 176 where IMAX is the number of preprogrammed configurations of stimulation signal generator parameters. Configure the stimulation parameters of the stimulation signal generator for iteration i. For example you may select a combination of pulse shape (e.g. monophasic or biphasic), pulse amplitude, pulse duration, pulse frequency, and duration of the application of the signal for each iteration. Note at this point a signal has not been connected to the output of the stimulation signal generator. Only when the instrument receives a start signal (either by manually pushing the start button or sending an electrical trigger start signal) will the output become active.

(step 163): Set N=42 (referring to the electrode 42)

(step 164): Connect electrode N with terminal 153

(step 166): Start recording the output of the monitor unit 66. After 5 seconds initiate stimulation

(step 168): After the duration of the stimulation signal is completed, the stimulation signal is turned off. However, the monitor unit output may continue to record past turn off time, T1.

(step 170): Once T1 has been completed, then the response time graph is calculated. The response is the difference between the measured baseline parametric value and the stimulated response. If the response meets predetermined criteria, for example the amplitude of the response is greater than 20% of the baseline, then electrodes 42 is a candidate for the Target Location and go to Step 174. Note that the predetermined criteria may be a function of the measured values, for example the root mean square (RMS) average over a time after start of stimulation.

(step 172): Disconnect electrode N from terminal 153. Set N=N+1. If N=50 (i.e., the last electrode 50) then go back to step 162. Go to Step 163.

(step 174): Discovery procedure successfully detects Target Location. Proceed to treatment procedure.

(step 176): Discovery procedure not successful. Proceed to self-diagnostic tests.

Case 2: Implementing Method 1 Using the Bipolar Option

Consider FIG. 22 as the schematic for an apparatus for implementing method 1 using the bipolar option. As discussed previously the apparatus can be configured to connect each of the electrodes on the catheter distal assembly to the terminals (153 and 154) of the stimulation generator 63. In this example, specific electrodes on the catheter distal assembly will be connected to either terminal 153 or 154. For this discussion, the sensor element 58 is measuring blood velocity and it is connected to monitor unit 66. The catheter distal assembly is positioned such that the electrodes 42 through 49 are in good electrical contact with the superior surface of the renal artery 59.

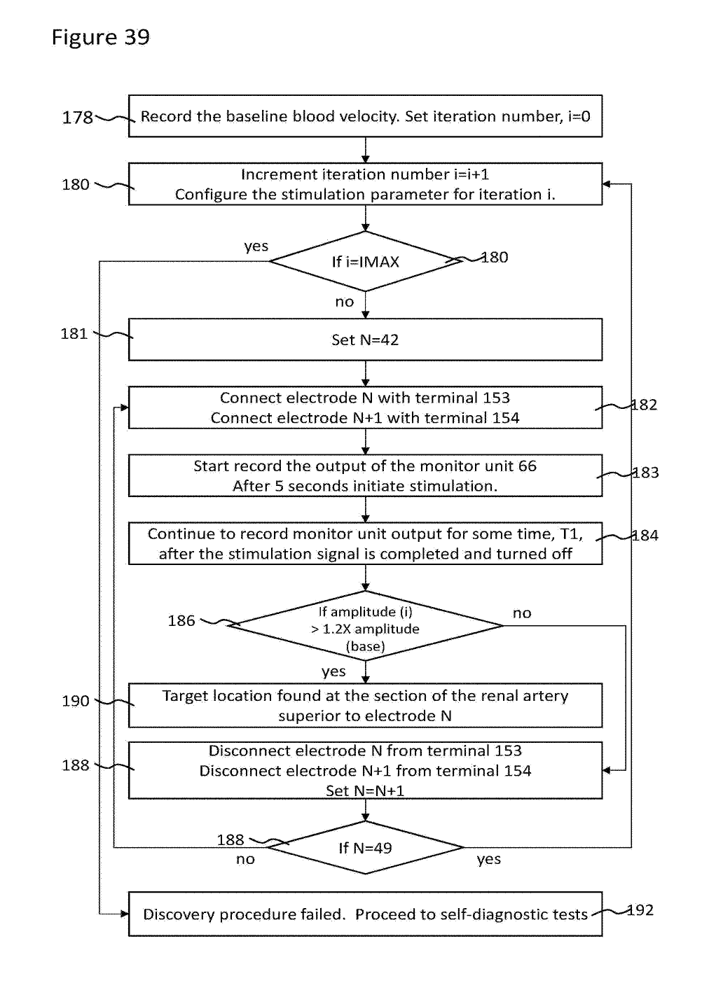

The following is an example of a search algorithm that searches for the Target Location. See FIG. 39 for a flowchart diagram of this algorithm. Start by connecting the two electrodes closest to the ostium to the stimulation signal generator (i.e. electrode 42 is connected to terminal 153 and electrode 43 is connected to terminal 154). Subsequent stimulations move the stimulation pairing by one electrode, e.g. the next pairing would be electrode 43 connected to terminal 153 and electrode 44 is connected to terminal 154. This process continues until either a stimulation response of sufficient magnitude is detected or the most distal electrode pairing has been tested. If none of the stimulation at electrodes pairings (42, 43), (43, 44), (44, 45), (45, 46), (46, 47), (47, 48) or (48, 49) elicit a strong enough response then the stimulation parameters are adjusted and the process is repeated. If a Target Location cannot be located after completing all sets of the stimulation parameters, then a diagnostic test must be run to determine the root cause of the failure of the discovery procedure.

Example of Search Algorithm:

(step 178): Record the baseline blood velocity prior to stimulation. Set iteration number, i=0.

(step 180): Increment iteration number i=i+1. If i=IMAX then go to step 192, where IMAX is the number of preprogrammed configurations of stimulation signal generator parameters. Configure the stimulation parameters of the stimulation signal generator for iteration i. For example, you may select a combination of pulse shape (e.g. monophasic or biphasic), pulse amplitude, pulse duration, pulse frequency, and duration of the application of the signal for each iteration. Note at this point the signal has not been connected to the output of the stimulation signal generator. Only when the instrument receivers a start signal (either by manually pushing the start button or sending an electrical trigger start signal) will the output become active.

(step 181): Set N=42 (referring to electrode 42)

(step 182): Connect electrode N with terminal 153. Connect electrode N+1 with terminal 154.

(step 183): Start recording the output of the monitor unit 66. After 5 seconds initiate stimulation.

(step 184): After the duration of the stimulation signal is completed the stimulation signal is turned off. However, the monitor unit output may still continue to be recorded past this turn off time for some time, T1.

(step 186): Once T1 has been completed, then the response time graph is calculated. If the response meets predetermined criteria, for example the amplitude of the response is greater than 20% of the baseline, then the Target Location is located between electrode N and electrode N+1. Confirmation of the discovery of the Target Location may be done by repeating this step. Then go to step 190.

(step 188): Disconnect electrode N from terminal 153 and disconnect electrode N+1 from terminal 154. Set N=N+1. If N=49 then go back to step 180. Otherwise go to Step 182.

(step 190): Discovery procedure successfully located Target Location. Proceed to treatment procedure.

(step 192: Discovery procedure not successful. Proceed to self-diagnostic tests.

Case 3: Implementing Method 2

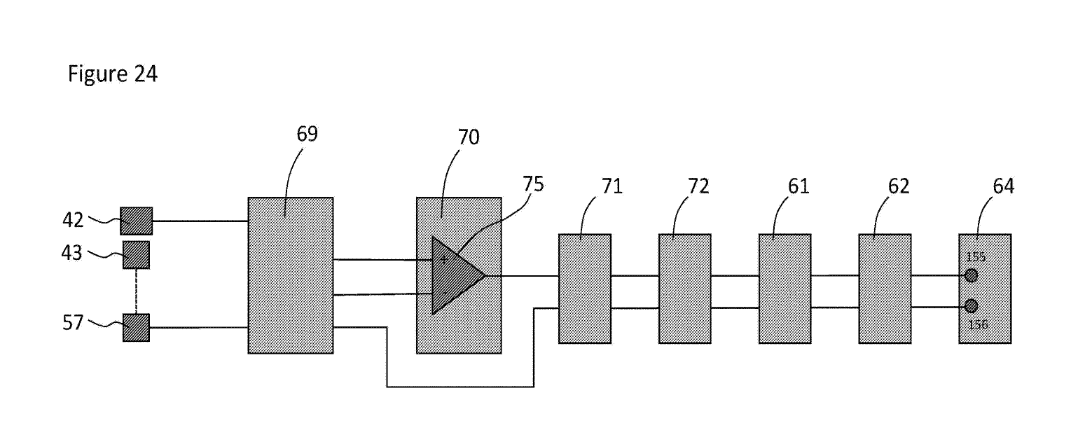

Consider FIG. 23 and FIG. 24 as the schematics for an apparatus for implementing method 2.

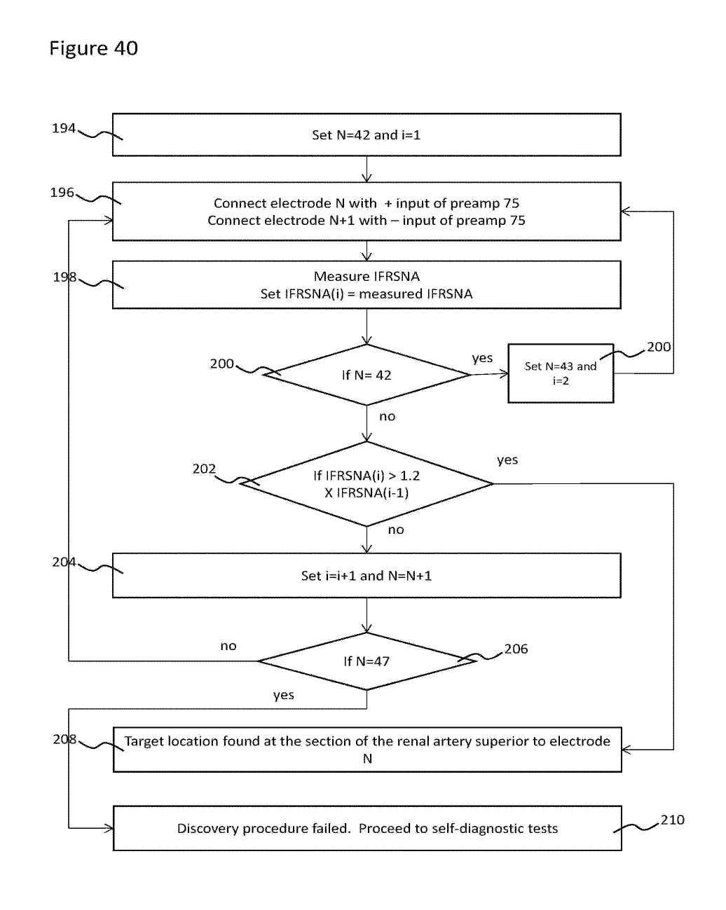

Measurement of RSNA (renal sympathetic nerve activity) at a location is accomplished by selecting two electrodes adjacent to that location. If no appreciable RSNA is measured, then no nerves are within the range of that the receiver. As shown in FIG. 3 the ARG 13 postganglionic nerves begin to innervate the renal artery directly inferior to the ARG. The section of the superior renal artery between the Target Location and the ostium has little or no innervation; therefore, electrodes adjacent to this section will detect little or no RSNA. Electrodes that are located distally from the Target Location will detect RSNA, but with a reduced amplitude due to dispersion of neural activity as the nerves branch out progressively towards the kidney. The example search algorithm shown (see FIG. 40 for a flowchart diagram of this algorithm) below measures RSNA for two sets of electrodes located on the superior side to detect a significant change in RSNA as the pairing is incrementally change from (42,43) to (43,44) to (44,45), etc. For a given pairing (i, i+1) the i electrodes are connected to the +input of the preamplifier 75 shown in FIG. 24 and the i+1 electrodes are connected to the −input of the preamplifier 75. The output of preamplifier is eventually connected to detector/receiver 64, via junction box 71 and junction box 72 and junction box 61 and cross connect module 62. Note that this is just one example of pairings. Another example is to choose pairings that are circumferential, for example for a given axial position you could use both the superior electrode and its complementary electrode, e.g. pairing electrodes 42 and 50, 43 and 51, 44 and 52, etc.

In order to determine the difference in RSNA it is necessary to process the signals that are received. One possible method is to first bandpass the RSNA signal from the detector/receiver, then integrate the area under the curve with the x-axis being time and the bandpassed RSNA signal as the y-axis for a fixed time duration. Call this quantity IFRSNA (Integrated filtered RSNA).

Example of Search Algorithm

(step 194): Set N=42, i=1; where N and i are numerical indices

(step 196): Connect electrode N with the +input of the preamplifier 75. Connect electrode N+1 with the −input of the preamplifier.

(step 198): Accumulate the IFRSNA for a fixed period time, e.g. 30 sec. Record this value as IFRSNA (i)

(step 200): If N=42, then set N=43 and i=2 and go to Step 196.

(step 202): If N>42, then compare IFRSNA (i) to IFRSNA (i−1). If IFRSNA (i) is greater than IFRSNA (i−1) by a predetermined % amount, e.g. 20%, then go to step 208.

(step 204): Set i=i+1 and N=N+1

(step 206): If N=47 go to Step 210, otherwise go to Step 196.

(step 208): Discovery procedure successfully locates Target Location. Proceed to treatment procedure.

(step 210): Discovery procedure not successful. Proceed to self-diagnostic tests

### Treatment Section

This section of the disclosure describes methods and apparatuses used to treat the ARG. As described previously in the disclosure, prior to initiating the treatment procedure, the discovery procedure should be completed to determine the Target Location. The Target Location can be used to determine a Target Volume, where the Target Volume is defined to be a volume that encompasses the aorticorenal ganglion. It is recognized that the target volume that encompasses the aorticorenal ganglion my also include parts of, or the entirety of other ganglia that are immediate proximity to the aorticorenal ganglion. For example, it is known that the renal inferior ganglion and the renal posterior ganglion are ganglia that are immediately adjacent to the aorticorenal ganglia. Patient to patient variations show measureable differences of the orientation and position of these ganglia relative the aorticorenal ganglia.

The objective of the treatment of the ARG is to alter the ARG to the extent that it is no longer functional, e.g. disable the ARG such that, for example, it can no longer transmit nerve signal and/or receive nerve signals and/or process nerve signals.

For heat-inducing treatment schemes, thermal sensing will be utilized in immediate proximity to locations where treatment energy first contacts patient tissue in order to assure a safe, controlled procedure. One method to achieve this is to have a thermal sensing component for each electrode. Another method that uses fewer sensors is to use a flexible printed circuit board to carry a group of electrode with a single thermal sensing component that has a low thermal resistance but high electrical resistance to each of the electrodes.

Treatment Modalities with Enhanced Radio Frequency (RF) Directionality

While RF ablation can be a further treatment option, what is further disclosed here are methods of directing RF ablation energy preferentially towards the ARG. Note devices may be implemented incorporating one or more of these methods. It is advantageous to be able to direct the RF energy so that a larger percentage of the energy input in the electrode catheter actually reaches the ARG and/or the volume immediately surrounding it. This allows the use of less energy to achieve the same treatment results, which in turn results in less collateral damage, e.g. damage to the renal artery wall and surrounding untargeted tissue.

The Following are Examples of Methods that May be Employed to Treat the ARG:

Method 1 (with Associated Apparatus with Usage Model)

Method 1 encompasses methods which use placement of the electrodes to more efficiently direct the RF energy to the ARG. One example is using an electrode catheter introduced into the renal artery 59 with electrodes that are positioned on the superior side of the renal artery wall.

It is known that in mammals, the ARG is generally located superior to the renal artery. We have confirmed this to be true in our animal studies, see Experiment #3

Consider FIG. 21a which shows the placement of the electrode 42 through 49 in contact with the superior side of the renal artery wall. For exemplary purpose, suppose the Discovery procedure has been completed and it has been determined that electrode 45 is the location of the Target Location. One can then treat the ARG using a monopolar technique or a bipolar technique. For the monopolar case, referring to FIG. 23, the cross connect module 62 is configured to connect electrode 45 with first terminal 157 of the RF electrical surgical generator 65. The second terminal 158 of the RF electrical surgical generator is connected to a dispersive pad attached externally to the back of the patient by configuring multiplexer unit 68 to select the appropriate dispersive pad lead from the return wire bundle 150.

For the bipolar case, there are a number of options. One option is to drive the RF ablation current through the two electrodes that are located on the same catheter distal assembly (bipolar-type1). For example, for this case, in FIG. 23 one could implement a bipolar-type1 procedure by selecting (via the cross connect module 62) electrodes immediately adjacent to the Target Location, i.e. using electrodes 44 and 46 when it is determined that electrode 45 is determined to be adjacent the Target Location. In this 811818-504 case the first terminal 157 of the RF electrical surgical generator would be connected to electrode 44 and second terminal 158 of the RF electrical surgical generator would be connected to electrode 46.

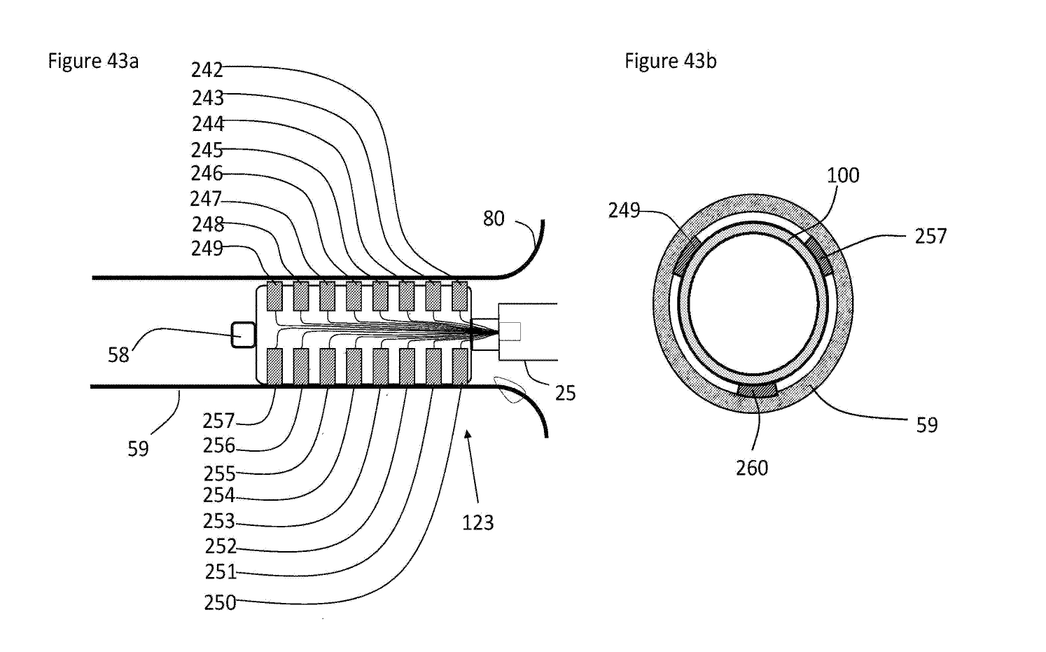

A second example of bipolar-type 1 is shown in FIG. 43a and FIG. 43b. The electrodes 242 through 257 are activated in pairs for either discovery stimulation or for treatment ablation. The electrode pairs are therefore (242,250), (243,251), (244,252), (245,253), (246,254), (247,255), (248,256) and (249,257). For example, if one considers the first pairing (242,250) in stimulation mode electrode 242 would be connected to the first terminal of the stimulation signal generator and electrode 250 would be connected to the second terminal of the stimulation signal generator. Furthermore, if one considers the first pairing (242,250) in treatment mode then electrode 242 would be connected to the first terminal of the electrical surgical generator and electrode 250 would be connected to the second terminal of the electrical surgical generator. A space element 260 may be included to provide symmetry to main uniform pressure against the renal artery wall.

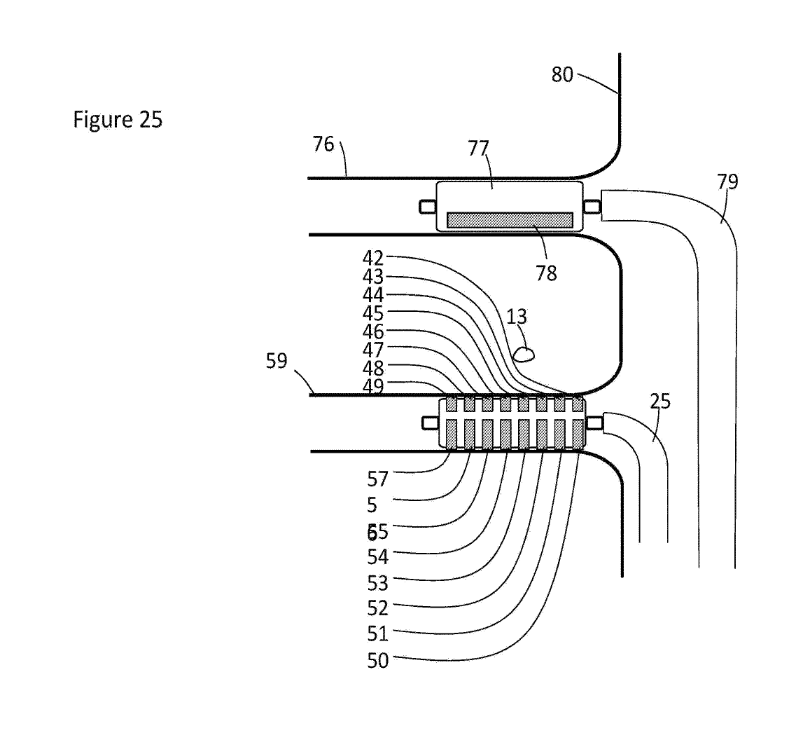

A second bipolar option, (bipolar-type2) is to connect first terminal 157 of the RF electrical surgical generator with electrode 45 and then connect second terminal 158 of the RF electrical signal generator with an electrode of a 2^{nd} catheter that is placed such that the ARG is roughly between electrode 45 and the electrode selected of a 2^{nd} catheter. Possible sites for location of the 2^{nd} catheter electrode are in the splenic artery (when the 1^{st} catheter is in the left renal artery) and the common hepatic artery (when the 1^{st} catheter is in the right renal artery), and the complementary aorta wall 80 superior to the subject renal artery. FIG. 25 shows an example of the 2^{nd} catheter inserted into the lumen of the common hepatic artery. Note that the 1^{st} catheter is inserted through guide catheter 25, with the second catheter inserted through the guide catheter 79. Both guide catheters are inserted through the Aorta. The second catheter distal assembly 77 only has one large electrode 78 which is in contact with the inferior side of the common hepatic artery wall 76. The important feature to note is that the ARG 13 is between the large 2^{nd} catheter electrode 78 and the 1^{st} catheter electrode 45. This ensures that the electric field that is produced when voltage is applied between electrode 45 and electrode 78 will be concentrated in the tissue containing the ARG, which in turn results in high efficiency delivery of the RF energy to the ARG.

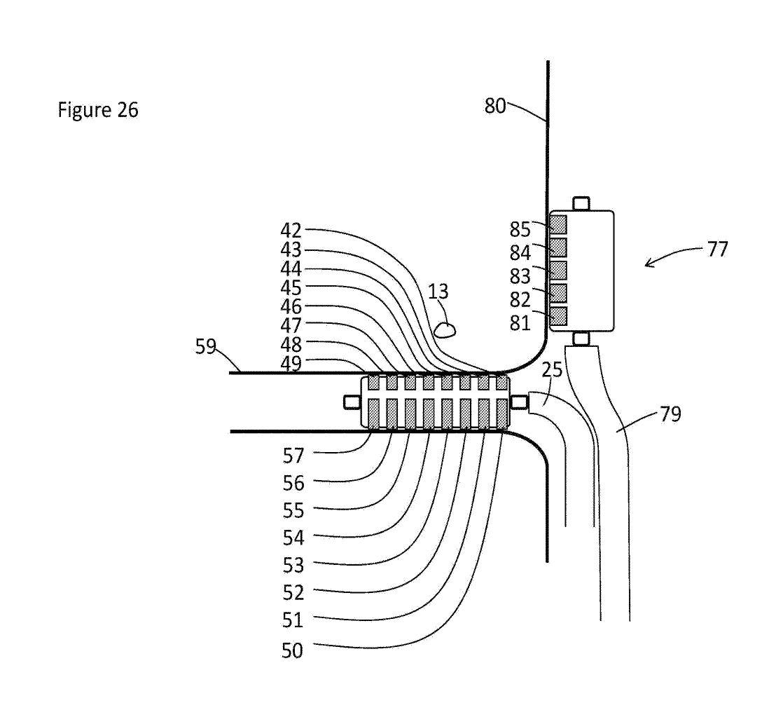

A second example of bipolar-type2 is shown in FIG. 26. In this Figure the 2^{nd} catheter with catheter distal assembly 77 is placed via guide catheter 79 such that its electrodes 81, 82, 83, 84, and 85 are in contact with the aorta's wall 80 just superior to the ostium of the renal artery 59. This apparatus configuration has several use cases of interest.

Case 1

Consider that there are several choices of pairing electrodes connected to the first terminal of the RF electrical surgical generator 157 with electrodes connected to the second terminal 158 of the RF electrical surgical generator that have the ARG roughly between each electrode pair. For example if the Target Location is located at electrode 45 then the following pairing will create an electric field of high intensity at the ARG: (46,85), (47,84), (48,83) and (49,82). To avoid high current densities at the aorta wall 80 or the renal artery wall 59 one may time division multiplex those parings such that one pairing is only on ¼ of the total time of treatment. This has the advantage of producing a higher average concentration of current in the ARG tissue while achieving a lower average current density at the aorta and renal artery walls.

Case 2

Alternatively, one could connect 82, 83, 84, and 85 to the second terminal 158 of the RF electrical signal generator and time division multiplex the connection to the first terminal of the RF electrical surgical generator 157 between 46, 47, 48, and 49.

Case 1 has a higher ratio of average current at the ARG to average current at the vessel walls (either aorta or renal artery walls) relative to case 2.

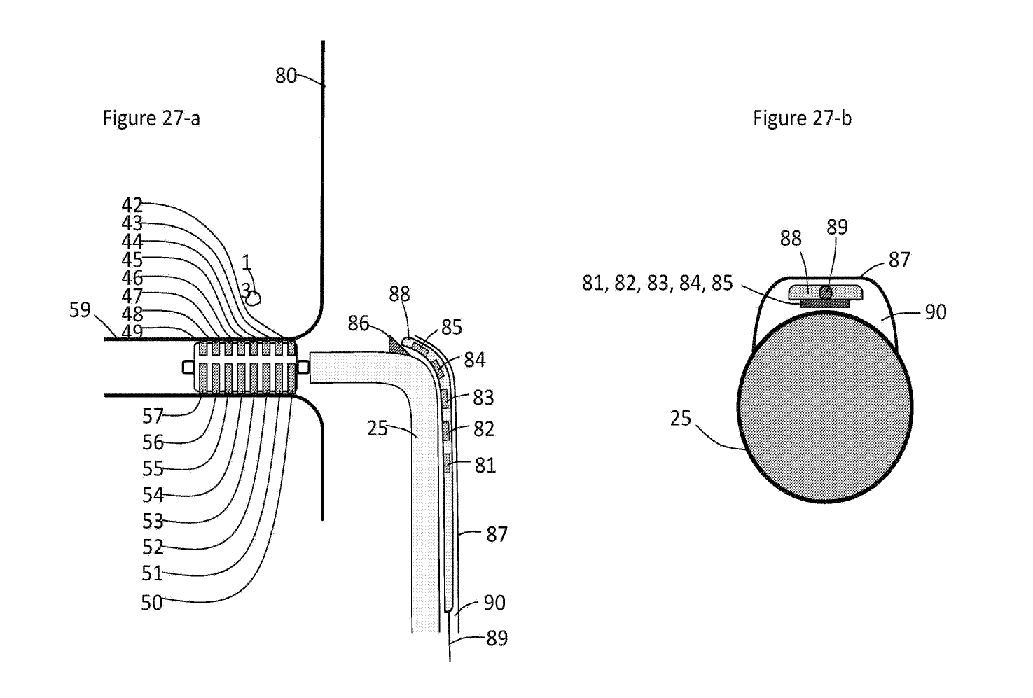

Bipolar-type2 configurations have the potential disadvantage of requiring 2 catheters as illustrated. One way to simplify the amount of interventional devices necessary to accomplish a bipolar-type2 configuration is to modify the guide catheter such that it has the ability to carry, deploy and actuate electrodes on a flexible circuit board. Consider FIG. 27-b which shows an additional channel 90 established by adding an additional wall 87 throughout the longitudinal length of a guide catheter 25. In the channel 90 a flexible printed circuit board 88 resides in the distal end of the cavity. The flexible printed circuit board 88 has electrodes 81, 82, 83, 84, and 85. The electrodes are connected to wires inside of a multipurpose cable 89. The second purpose of the multipurpose cable 89 is to act as an actuator cable for deploying and extracting the flexible printed circuit board via its attachment point 91.

Feature 86 is a component that performs as an inclined plane or ramp in its deployed state by directing the advanced printed circuit board 88 in the general direction of the aorta wall 80 superior to the renal artery 59.

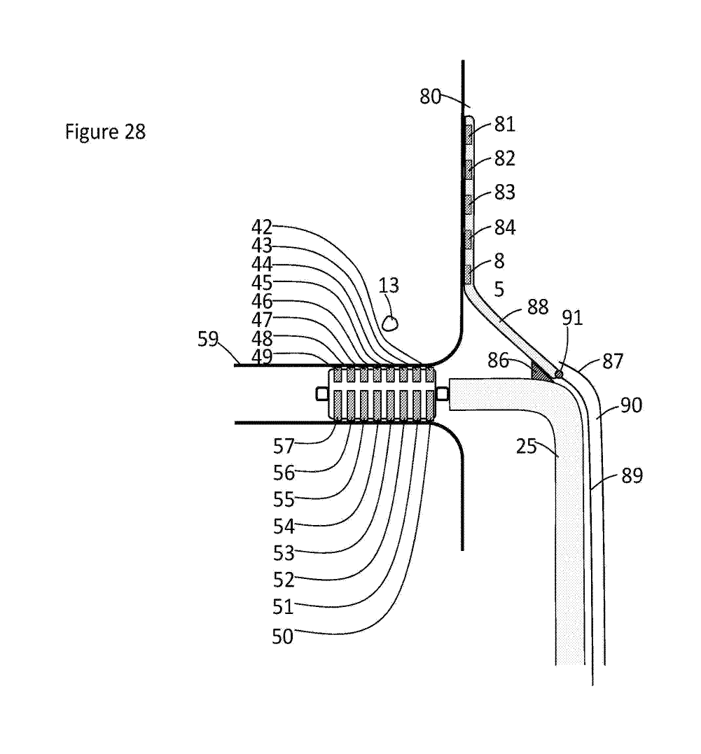

FIG. 28 shows the catheter system with its printed circuit 88 board fully deployed and its electrodes 81, 82, 83, 84, and 85 in full electrical contact with the aorta wall 80. Note that good electrical contact can be achieved by a number of methods, one of which is a leaf spring on the back of the printed circuit board. Another method is to mount the printed circuit board on a polymer substrate that can change its rigidity as electrical voltage is applied (e.g. electroactive polymers).

Method 2

Method 2 encompasses methods that are used to electrically isolate the source electrodes from the blood. It is known that blood has a higher electrical conductivity than most types of body tissue (C. Gabriel, et al, Physics in Medicine and Biology, 54, 2009: 4863-4878). Therefore, in the absence of electrical isolation of treatment electrodes from the blood stream, a significant portion of the RF energy delivered into the patient circumvents the tissue volume containing the ARG, instead being conducted by the blood stream directly from one electrode to the other of opposite potential. This has been shown experimentally to effect energy penetration and directionality, potentially diminishing efficacy of treatment, while increasing risk of collateral damage of non-targeted tissue and potentially introducing procedural complications by exciting undesired sensory responses.

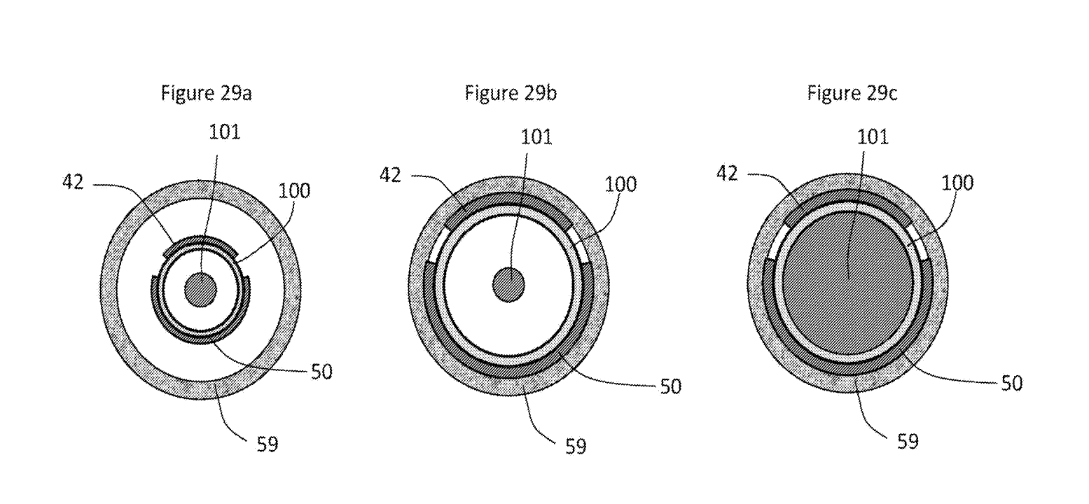

FIGS. 29a, 29b, and 29c show the cross section of a catheter with the capability of isolating its electrodes from the blood by deploying a hydraulic balloon. FIG. 29a shows the catheter distal assembly in its unexpanded state in the renal artery 59. In FIG. 29b a mechanical construct 100, e.g. a mesh, basket, etc., changes the radial aspect of the catheter distal assembly, engaging the electrodes 42 through 57 with the renal artery wall 59. In FIG. 29c a balloon 101 is inflated with a gas or liquid such as saline so that outward expansion exerts circumferential pressure on the electrodes so that they are compressed into the renal artery wall 59 while simultaneously displacing blood from the conductive path. This optimizes electrical isolation of the electrodes 42 through 57 from the blood. Since the balloon blocks the blood flow in the renal artery when deployed, it is only inflated just prior to treatment, e.g. after discovery has been completed and then deflated immediately after treatment. The balloon can also employ a radiant cooling mechanism (e.g. circulated chilled saline as distension fluid) to protect the renal artery wall during thermal treatment.

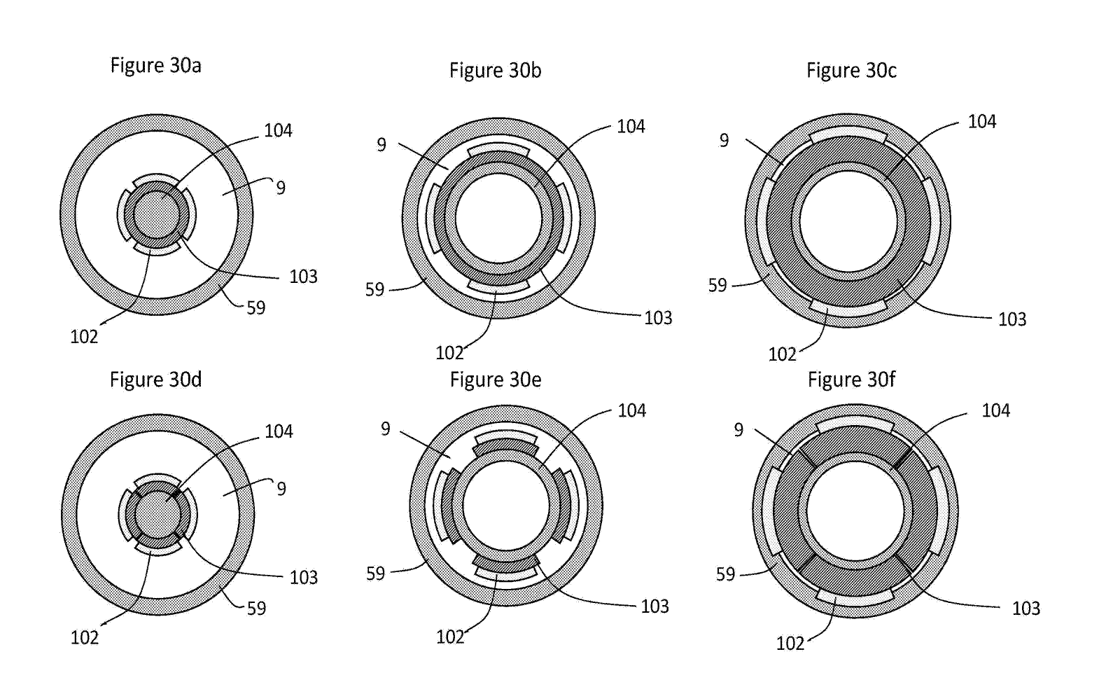

FIGS. 30a, 30b and 30c show an apparatus that provides electrical path isolation from blood, while simultaneously allowing central renal artery blood flow.

FIG. 30a shows a cross sectional view of the artery and the catheter distal assembly inserted into the renal artery 9. FIG. 30b shows a cross sectional view of the artery and catheter distal assembly after the mechanical construct 104 (e.g. basket construct, hoop construct, etc.) has been expanded. Note that the interior of this mechanical construct 104 is open and allows blood to flow. FIG. 30c shows a cross sectional view of the artery and catheter distal assembly with both the mechanical construct 100 expanded and the balloon 103 inflated. In FIG. 30c the electrodes 102 are compressed into the renal artery wall 59.

As previously described, blood is displaced from the renal artery walls thus isolating the electrodes and electrical path.

One method of constructing balloon 103 is to employ multiple longitudinal balloon sections. FIGS. 30d, 30e, and 30f show longitudinal balloons located between each electrode 102 and the mechanical construct 104. When inflated, the balloons join to fill the annular space between the mechanical construct 104 and the renal artery wall 59, driving electrodes into the renal artery wall 59 and vacating the blood immediately adjacent to the electrodes 102.

### Treatment Modalities Using RF Needle Based Electrodes

FIG. 8a, FIG. 8b, and earlier related portions of this specification describe the use of a needle coupled to the terminals of an electrical surgical generator. This section further details this bipolar approach.

In the bipolar case, one terminal of the electrical surgical generator is connected to the electrode needle while the other terminal is connected to another conducting element that is in close proximity.

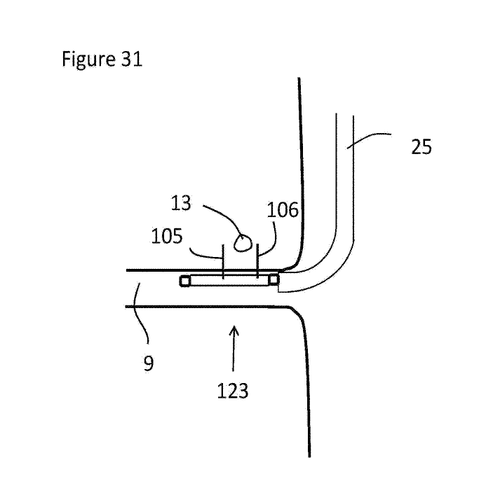

Examples implementations are: 1) Consider FIG. 31 which has a first electrode needle 105 and a second electrode needle 106 from the same catheter. In an optimal situation the conducting tips of the needles would be arranged such that the ARG is between them. Finding the optimal placement could use a combination of the discovery protocols to find the Target Location in the renal artery, followed by a second search to determine the distance superior to the Target Location where the ARG is located. The vertical discovery protocol would be similar to the discovery protocol used along the axis of the renal artery, i.e. using electrical stimulation in combination with parametric response measurements or RSNA to determine the Target Location.

2) a second electrode is located on a 2^{nd} catheter or guide catheter positioned in a nearby artery, such that the ARG is between said electrode and the needle from the 1.sup.st catheter.

3) Another bipolar configuration involves a single needle containing two isolated electrodes. The needle can be inserted into the tissue such that a conductive path is in proximity to the ARG.

It is also possible to introduce one or both of these electrode needles percutaneously without going through the vascular system, by directly inserting needles through the skin to the Target Location while monitoring the placement with an imaging system, e.g. a fluoroscopic imaging system, to avoid inadvertent contact with arteries, veins, etc.

### Treatment Modalities Using Lasers

The methods, apparatuses, and usages proposed in this section use laser energy to create a thermal ablation of the ARG. Laser-induced thermotherapy, also referred to as laser ablation, consists of tissue destruction, induced by a local increase of temperature by means of absorbing laser light energy transmission in the Target Volume, i.e. a volume encompassing the ARG.

Proper selection of the wavelength is important. The criteria for preferably wavelength selection are:

High absorption optical cross-section in the targeted tissue, i.e. the ARG

Lower absorption optical cross-section for the renal artery wall tissue

Low scattering optical cross-section for all tissue between the catheter and the ARG. Scattering of light not only reduces the amount of light reaching the target but also results in the dispersion of the incident beam into undesirable areas.

Irreversible necrosis of tissue irradiated by laser energy occurs as a combination of the temperature rise produced locally and the exposure time: cell death occurs within few seconds for temperatures exceeding 60° C., while for lower temperatures, the necessary exposure time is longer.

The advantage of using laser light for thermotherapy, compared to other methods, is its ability to deposit a precise amount of energy in a well-defined region. This is equivalent to saying that a laser based ablation system has the capability of defining beams of a given shape and size. This capability is enabled in large part because of two important properties of laser emissions: (1) the emitted light has a narrow spectrum, typically in the neighborhood of 1 nm or less, (2) the light emitted by a laser has a low etendue (a property of light in an optical system, which characterizes how constrained the light is in area and angle).

Most of the absorbed light is converted into heat, which causes changes in optical properties of tissue. Coagulation is defined as the thermal damage of the tissue proteins at temperatures in the interval between 55 and 95° C. Its extension region depends mainly on the time during which the temperatures remain within the range.

Method for Using Lasers to Treat the ARG

Method 1

In this method the laser source is external to the body. The laser source is coupled to a fiber or fibers and those fibers are routed through the elongated catheter body to its distal end and terminated in catheter distal assembly.

There are a number of reasons for keeping the laser external: (1) a set of lasers in a disposable catheter will be expensive, (2) laser are not very efficient and therefore will create a large thermal load to manage.

Apparatus for Using Lasers to Treat the ARG

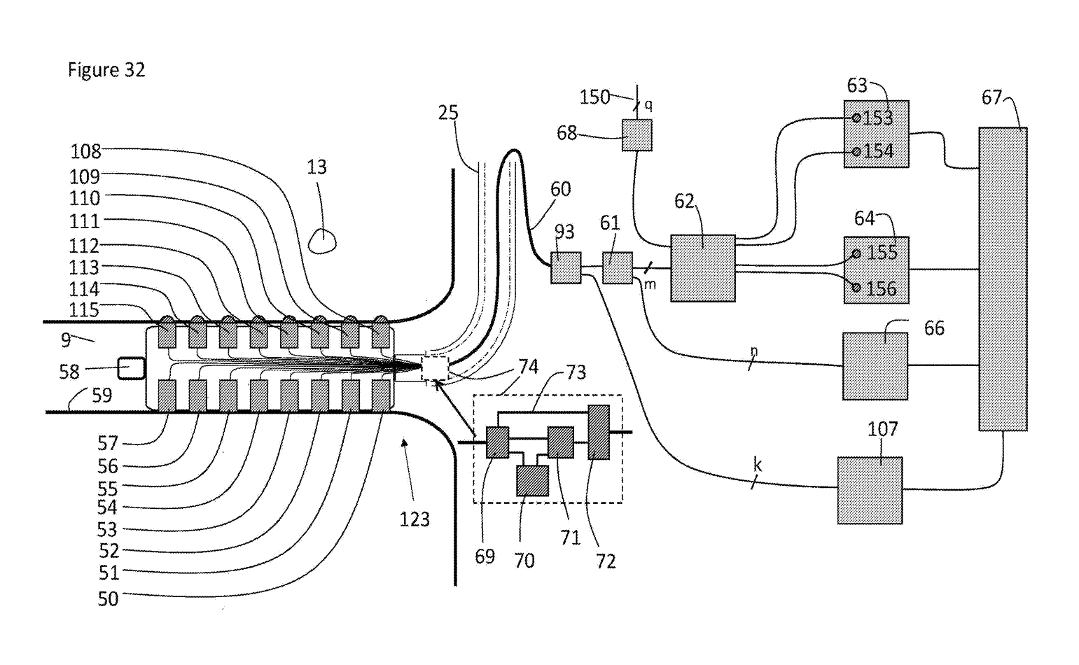

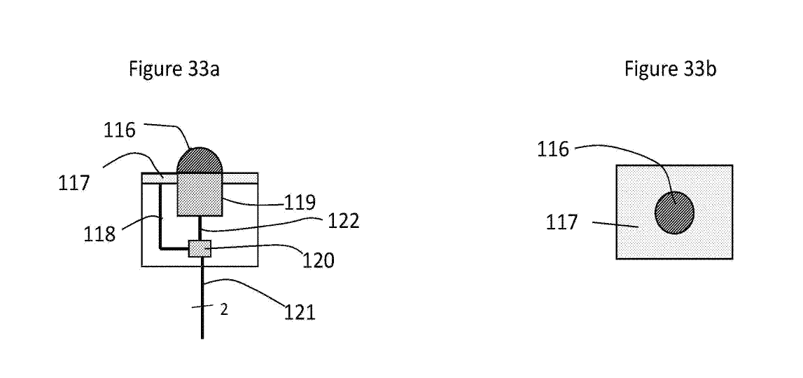

FIG. 32 shows the connectivity between the catheter distal assembly 123 elements and the external (outside the body of the patient) elements. Elements 108 through 115 are hybrid electrode/fiber optic units. As shown in FIG. 33a (side view) and FIG. 33b (top view) those elements contain a conductive electrode 117 and a fiber optic terminating/lensing system 119. Conductive electrode 117 is connected to wire 118 which is combined into a two element cable 121 via the junction box 120. The other element combined into this two element cable 121 is the fiber 122 that carries the light sourced by the laser source unit 107. The fiber 122 connects to the fiber optic terminating/lensing system 119. The light emitted from the end of the fiber is groomed into the desired beam shape via an optical lens unit 116. The electrical wires are externally separated/combined with the fibers in junction box 93, as shown in FIG. 32. The laser source unit 107 has the ability to couple its laser to any of the k fibers. FIG. 33b shows a top view of the elements 108 through 115. Each of these elements has an optical lens unit 116 surrounded by a conductive electrode 117.

Usage model apparatus shown in FIGS. 32, 33a, 33b.

A discovery procedure is executed to find the Target Location in exactly the same manner that was used in Discovery Section of this patent application by selectively applying stimulation to the electrode elements of the appropriate hybrid elements (108, 109, etc.). Once the Target Location is located then the laser source unit 107 is coupled to the appropriate fiber and the laser is activated for the designated treatment period. For example, suppose that the electrode in hybrid element 111 is determined to be the Target Location. Then the fiber attached to hybrid element 111 is selected by the output of the laser source unit 107. Because the ablation is optical, rather than electrical RF, it is possible that one could measure parametric responses during the stimulation and determine when to terminate the application of laser power once the desire parametric response had been achieved.

### Treatment Modalities Using Ultrasound

An aorticorenal ganglion modifying catheter employing ultrasound energy for tissue modification may utilize piezo-electric crystal source(s) in a beam-focusing configuration as the tissue modifying element, coupled to an external energy source. Ultrasound energy in a wide range, for example from 10 KHz to 20 MHz, may be applied to affect tissue modification.

One embodiment is to adapt the catheter to enable High-Intensity Focused Ultrasound (HIFU) therapy, in which ultrasound beams are focused on the area surrounding the ARG, causing the targeted tissue temperature to heat to a range of 65°-85° C., thus modifying the ARG.

A potential benefit to ultrasound ablation is the simultaneous use of the same equipment for sensory imaging, thus providing real-time feedback of the state of tissue coagulation for control and safety.

Methods of Focusing and Directing Ultrasonic Power

Method 1

Method 1 uses the geometry of an assembly of reflective surfaces to create a focused beam. This assembly can then be steered and moved to direct the focused beam to the ARG.

Method 2

Method 2 uses a phased array of transducers to direct and focus the ultrasound radiated energy. By dynamically adjusting the phase and magnitude of the electronic signals to each of the elements of a phased array, the beam can be steered to different locations and focused.

Apparatus for Using Ultrasound to Treat the ARG

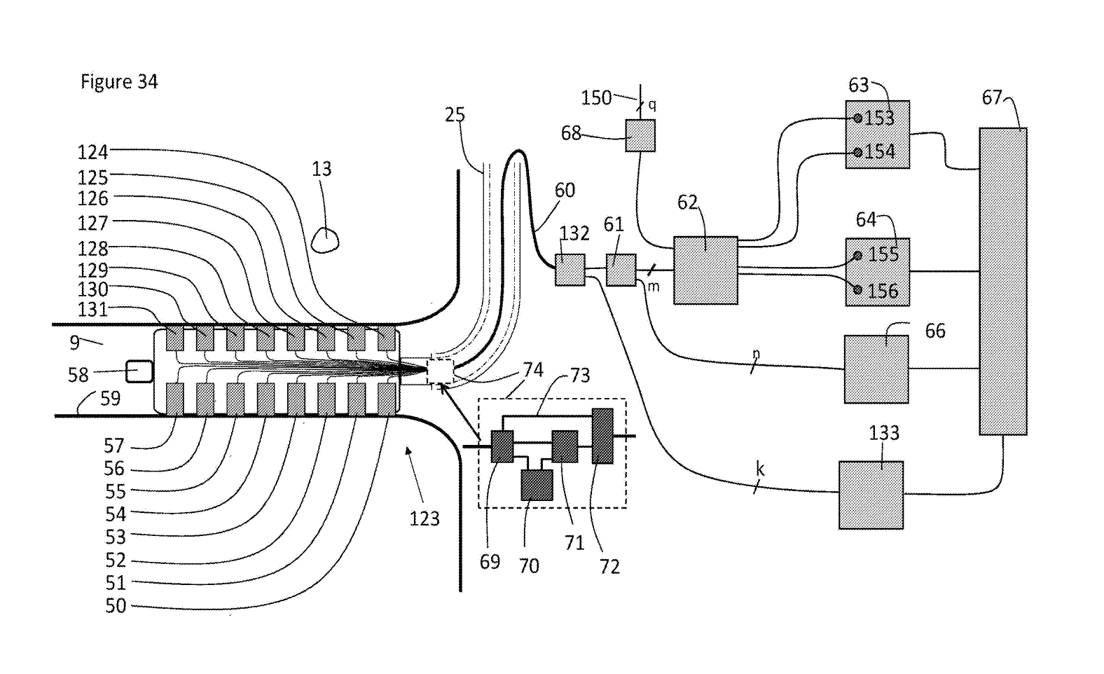

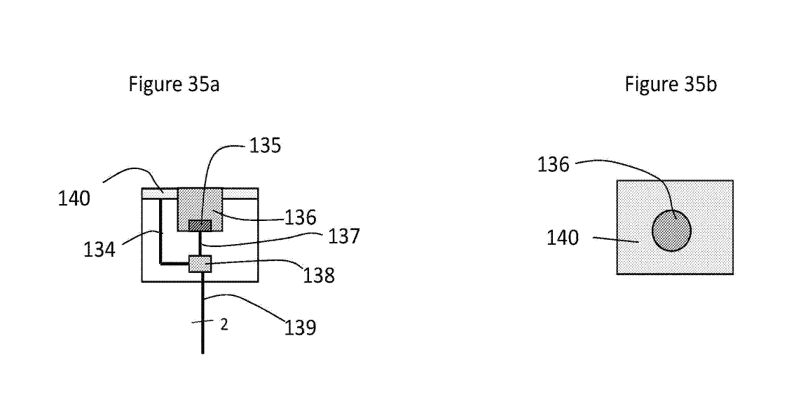

The apparatus shown in FIGS. 34, 35a and 35b utilizes the phased area method to focus the ultrasonic beam in conjunction with collimated piezoelectric element, where a collimated piezoelectric element confines the output to a conical shape. FIG. 34 shows an ultrasonic generator 133 supplying the phase delayed signal to hybrid electrode/piezoelectric elements 124 through 131. As shown in FIG. 35a (side view) and FIG. 35b (top view) those elements contain a conductive electrode 140 and a piezoelectric ultrasound source 135 and collimator 136. Conductive electrode 140 is connected to wire 134 which is combined into a two element cable 139 via the junction box 138. The other element combined into this two element cable 139 is the wire 137 that carries the electronically phased delayed signal from the ultrasonic generator 133.

Usage Model for Apparatus Shown in FIGS. 32, 33a, 33b

A discovery procedure is executed to find the Target Location similar to what was described in the Discovery Section of this patent application by selectively applying stimulation to the electrode elements of the appropriate hybrid electrode/piezoelectric elements (124, 125, etc.). Once the Target Location is located, the ultrasonic generator generates the appropriate phase delayed signal to focus and direct a beam of ultrasonic power 2 mm to 10 mm superior to the Target Location.

Monitoring ablation progress and maintaining directional and efficacy control during the HIFU procedure may rely on diagnostic techniques such as magnetic resonance imaging (MRI), fluoroscopic imaging, and ultrasound imaging.

Treatment Modalities Using Microwaves

The methods, apparatuses and usages described in this section use microwave energy to create a thermal ablation of the ARG.

One way to accomplish this is to use an Aorticorenal ganglion modifying catheter which creates a focused beam of microwave energy and furthermore can steer that beam to the ARG location.

The apparatuses disclosed herein also have the ability to discover the location of the optimal location for treatment and to confirm if that the treatment was successful.

Methods of Focusing and Directing Microwave Radiation

Method 1

Method 1 uses a physical construct, e.g. a parabolic reflector which reflects the microwave energy from a microwave emitter element(s) to create a focused beam. In addition, a mechanical means is supplied to direct the beam created by the parabolic reflector to the ARG location. This method has the advantage that it uses a single transmission line (in combination with power splitters if multiple emitters are used)

Method 2

Method 2 uses multiple microwave transmission emitter elements that are arranged in a linear array. Each of the microwave transmission emitter elements are connected to transmission line which in turn is connected to a port of a microwave generator. Each port of the microwave generator sources microwaves at the same frequency but with a programmable phase delay. By changing the phase delay and amplitude, the direction and focus of the beam can be changed. This method has the advantage that the beam can be steered without moving parts. It also has the advantage of a lower incidence of intensity of microwave power on the renal artery wall because multiple sources are employed.

### Apparatuses and Usage for Implementing Aforementioned Methods

Apparatus and Usage for Implementing Method 1

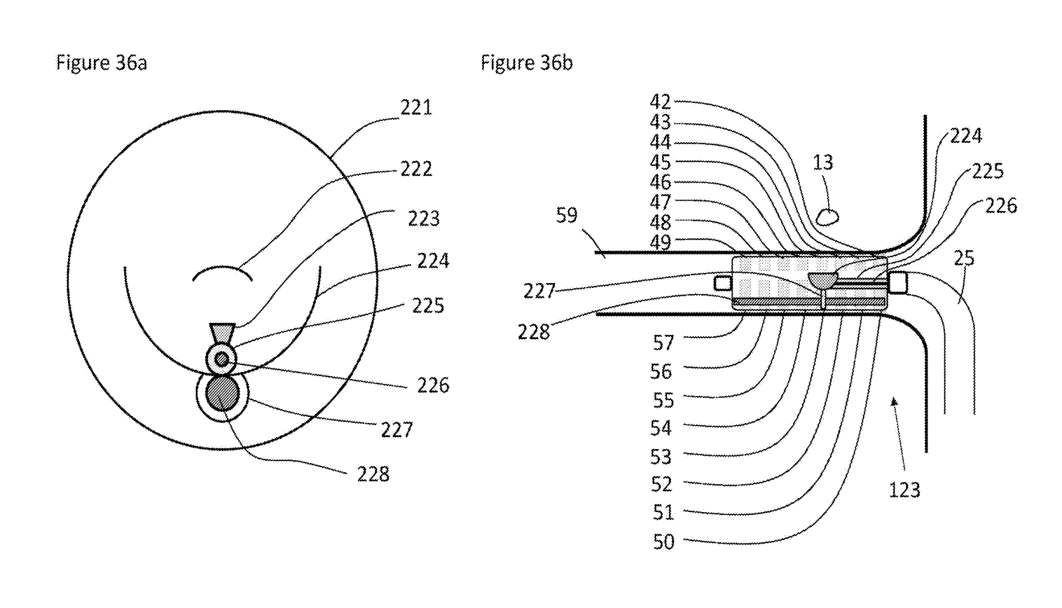

FIG. 36a represents a radial cross section and FIG. 36b represents a longitudinal cross section of an ARG modifying catheter for employing method 1. Elements 222, 223, 224, and 1227 are connected to form an assembly which is free to move axially and rotationally along the length of shaft 228. The microwave antenna 223 converts the microwave conducted current into microwave radiation. The microwaves emitted by the antenna 223 are directed towards a first metal surface 222 which in turn reflects these waves to a second metal surface 224. The waves reaching the second metal surface are directed into a beam. Consider that elements 222, 223, 224, and 227 are part of sub-assembly that can move as a unit. The beam is steered by coupling this sub-assembly (of elements 222, 223, 224 and 227) to a shaft 228 via coupling element 227. The aforementioned sub-assembly is attached to control cable 225 and may be retracted or extended and/or rotated via manipulation of a control cable 225. Stress relief of the transmission cable 226 is accomplished by attaching the transmission cable 226 to the control cable 225. The usage model would control the input microwave power while the parabolic antenna is being positioned or re-positioned. Reflected power is monitored and must meet safety criteria before high power can be applied. For example, high reflected power could cause standing waves which might endanger the patient.

Electrode elements 42 through 57 are still present for purposes of discovering the Target Location and for confirming effectiveness of treatment. Once the Target Location has been discovered, then the beam formed by the aforementioned sub-assembly may be directed towards the ARG by changing the subassembly's axial location and/or its angle relative to the axis of the shaft 228 by manipulating the control cable 225.

To decrease the amount of reflected microwave energy from electrode elements 42 through 49 the electrode's metallization may be cross hatched and/or they may be backed by a microwave absorbent coating.

The location of the electrodes and the location of the center of the microwave source assembly are enabled by radio opaque markers.

An advantage of the focusing aspect of this implementation is that intensity of the microwave power will decrease as the distance from focus increases. Therefore, tissue that is beyond the ARG will be subjected to less intense microwave power, resulting in less collateral tissue damage.

Apparatus and Usage for Implementing Method 2

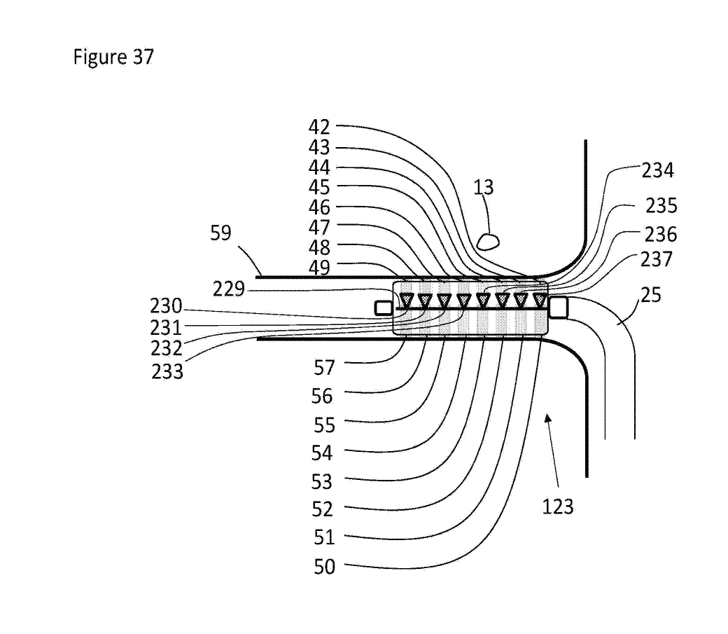

FIG. 37 represents a longitudinal cross section of an ARG modifying catheter employing method 2. Elements 230 through 237 are microwave emitters each of which is attached to its own microwave transmission line. Element 229 represents eight microwave transmission lines, successively terminated in elements 230 through 237. The other end of each microwave transmission line is attached to a port of a microwave generator. The amplitude and phase delay of each port is adjustable. This enables the microprocessor controlling the system to adjust the phase and amplitude to steer the focus of the beam without physically moving the elements. An alternative approach is use a single microwave transmission line and a set of individually programmable phase delay elements in the catheter distal assembly. This has the advantage of reducing the amount of microwave transmission cables and therefore makes the elongated catheter body more flexible.

### Confirmation Section

This section describes methods and apparatuses used to determine if the treatment of the ARG and/or the post-ganglionic nerves has been successful. Successful treatment is defined as modifying the ARG and/or the associated ARG post-ganglionic nerves such that the nerve signals can no longer be transported and/or processed by the ARG and/or transported by the ARG post-ganglionic nerves.

The Following are Examples of Methods that May be Employed to Confirm Successful Treatment:

Note that in the methods described below it is necessary to first establish pre-treatment measurements.

In the case where the measurement is a parametric response (e.g. renal artery blood flow) to stimulation (e.g. electrical stimulation), it is necessary to determine the pre-ablation response. The treatment is determined to be successful if the post-ablation response is measurably reduced compared to the pre-ablation response.

In the case where the behavior is characterized directly by a parameter, e.g. measuring renal sympathetic nerve activity, then it is only necessary to record the baseline pre-ablation parametric behavior. The success of the treatment is then determined by the relative comparison between the pre-ablation and post-ablation parametric behavior.

Method 1

Measure a change in a parametric post-treatment response (e.g. renal artery blood flow, renal artery blood velocity, renal artery diameter, etc.) to electrical or mechanical stimulation of the ARG and/or the ARG post ganglionic nerves.

The following are specific examples of this category of methods:

Renal Artery spasms can be induced by using a probe to put mechanical pressure on the renal artery wall. A reduction in responsivity of the renal artery to endovascular mechanical stimulation is an indicator that renal afferent nerves and/or the ARG have been compromised and are unable to transfer receptor signals to the CNS.

Researchers (P. Gal, et al, Journal of Hypertension 2014: 1-4) have reported changes in systemic blood pressure and/or heart rate, by applying electrical stimulation to the afferent nerves surrounding the renal artery. A reduction of responsivity in systemic blood pressure and/or heart rate to endovascular electrical stimulation along the renal artery is an indicator that the renal afferent nerves and/or the ARG have been compromised and are unable to transfer receptor signals to the CNS.

Endovascular electrical stimulation of the ARG and/or renal nerves can result in a number of measureable responses depending on the nature of the stimulation signal, (i.e. pulse shape, pulse amplitude, pulse frequency, pulse duty cycle, mono-phasic vs biphasic, etc.). The responses include contraction of the renal artery; change in the renal artery blood flow; change in the renal artery blood velocity, pulsations of the kidney.

Endovascular electrical stimulation of the ARG or renal afferent nerves can result in a number of measureable responses including the contraction of the renal artery at or near the site of stimulation and/or change in the renal artery blood flow, and/or change in the renal artery blood velocity. As shown in experiment #3 (below) with biphasic signals 7.5 volts in amplitude, the response is strongest for 20 Hz and 50 Hz frequency, with little or no response at 5 Hz. This would indicate that the response is initially triggered by inducing a signal in the afferent nerves. If the ARG or its associated afferent nerve bundles have been damaged then the aforementioned responses will be reduced or entirely absent.

Post ablation stimulation at the site of the ablation may cause a strong response in some cases for a number of reasons; e.g. the range of the stimulation range may exceed the treatment range, the local chemistry of the treated region has been altered by the treatment process, etc. An alternative method which avoids these issues is to apply the endovascular electric (or mechanical) stimulation distal to the site of the treatment. For example, if the treatment was applied at 9 mm from the ostium then the post ablation endovascular electrical stimulation could be applied at sites greater than 9 mm (e.g. 15 mm) from the ostium.

Method 2

Measure a change in a stimulated renal nerve transmission performance (e.g. conduction velocity, signal shape, signal amplitude, signal frequency).

Method 3

Measure a change in RSNA (Renal Sympathetic Nerve Activity) (e.g. signal shape, signal amplitude, signal frequency) without application of stimulation.

### Apparatus for Implementing Aforementioned Confirmation Methods

Refer to the apparatuses previously described in the Discovery Section to implement aforementioned confirmation methods.

Example of Use Cases of Apparatus:

Case 1: Implementing Method 1: Confirmation of Treatment by Stimulation of the Afferent Nerves

Let us suppose that an ablation procedure was performed using electrode element 44. Confirmation of said ablation can be accomplished by applying an afferent stimulation signal at various electrode elements distal from the site of the ablation (as mentioned earlier it is advisable to stimulate in locations that are distal from the original ablation site to avoid false negatives for success of treatment). This is done by programming the cross connect module 62 to make the appropriate connection between the stimulation signal generator 63 and the desired electrode element wires. The afferent stimulation signal should be approximately in the range of 20 Hz to 50 Hz. Electrode elements 46, 47, 48, 49, 54, 55, 56 and 57 are likely to be effective. Prior to ablation, baseline responses must be recorded. Post-ablation afferent stimulation responses can be measured for each electrode element. Alternatively, the electrode elements may be paired by axial location, e.g. 46 with 54, 47 with 55, 48 with 56 and 49 with 57. This may reduce the time for the completion of the confirmation test. If the post ablation response is significantly less than the pre ablation response for all electrode elements or electrode element pairs then it is an indication that the treatment was successful.

Case 2: Implementing Method 2: Measuring Change in a Stimulated Renal Nerve Transmission Characteristics (e.g. Conduction Velocity, Signal Shape, Signal Amplitude, Signal Frequency)

First assume that the ablation procedure was performed using electrode element 48. The success of the ablation can be determined by endovascular stimulation of the nerves distal from the site of the ablation and measuring the stimulated signal at the ablation site or proximal to the ablation site. The stimulated signal could be either an “afferent stimulation signal” or an “efferent stimulation signal”, with the primary difference being pulse frequency. In this test the stimulation signal generator is sequentially connected to aforementioned axial electrode element pairs, e.g. starting with electrode elements 42 and 50, then proceeding to electrode elements 43 and 51, then proceeding to electrode elements 44 and 52 and finally to electrode elements 45 and 53. In each case the detector/receiver unit 64 is connected to electrodes 48 and 56. The comparison of the pre-ablation stimulation response (signal shape, signal velocity, signal amplitude, etc.) with the post-ablation stimulation response will determine if the treatment was successful.

Case 2: Implementing Method 3: Measuring a Change in RSNA (Renal Sympathetic Nerve Activity) without Application of Stimulation

Assume for this case that the ablation procedure was performed using electrode element 44. Normal operation of the renal complex requires constant signaling to take place on the efferent and afferent nerves that innervate the renal artery walls and the surrounding tissue. The nature of that traffic is significantly altered if the ARG or the associated postganglionic nerves have been altered. This can be observed by sequentially connecting electrode elements to the detector/receiver 64 and recording the RSNA before and after ablation. The magnitude of the change of the post-ablation RSNA versus the pre-ablation RSNA is a metric that can be used to determine if the ablation was successful.

As mentioned in the Discovery Section of this disclosure, RSNA is a function of time. In order to achieve a figure of merit to determine the difference in RSNA (pre-ablation versus post-ablation) it is necessary to process the signals that are received. One possible method is to first bandpass the RSNA signal from the detector/receiver, then integrate the area under the curve with the x-axis being time and the bandpassed RSNA signal as the y-axis for a fixed time duration. This quantity is referred to as IFRSNA (Integrated filtered RSNA).

### Mesh Catheter Section

In accordance with an aspect of the current invention, an aorticorenal ganglion modifying catheter comprises an elongated catheter body extending longitudinally between a proximal end and a distal end along a longitudinal axis and a mesh element assembly connected to the catheter distal assembly comprising radiofrequency electrode elements attached to outer surface of mesh element. Mesh element has a proximal end connected to catheter distal assembly and a distal end. Mesh element is movable between a collapsed configuration and an expanded configuration. When mesh element is in proximity of aorticorenal ganglion, mesh element is expanded allowing for tissue contact with the radiofrequency electrode element. Ganglionic tissue modification is achieved as previously described with monopolar and bipolar electrode element catheters.

Description of the Invention

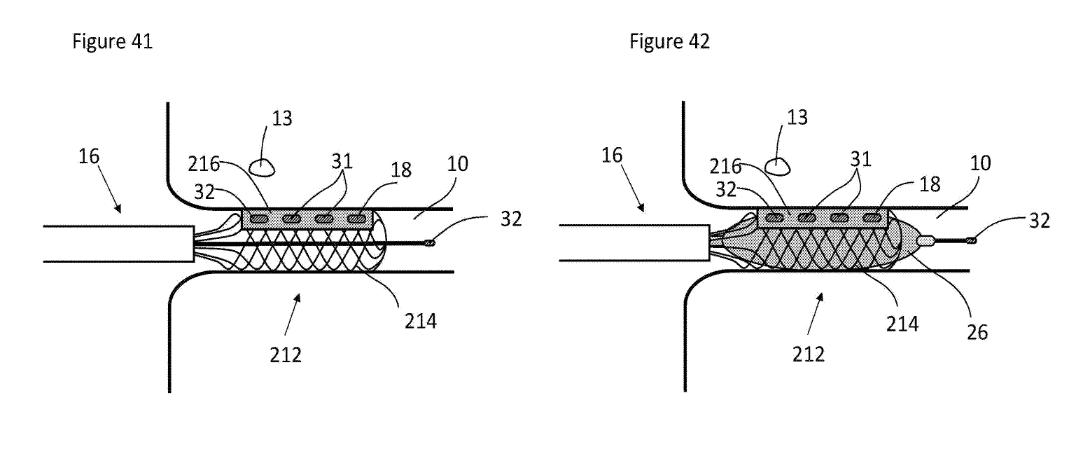

A similar device to the catheter assembly 16 of FIG. 7 is shown in FIG. 41. Basket element assembly 26 is replaced with a mesh or braid element assembly 212. Mesh element assembly 212 comprises interwoven or intertwined wired structure 214. Mesh material can be manufactured with an assortment of deformable materials including metallic wires (e.g. steel and nitinol), insulated metallic wires and semi-rigid plastics (e.g. nylon, fluoropolymers, etc.). Tissue modifying element 18 may be directly attached to the mesh element. Mesh element assembly 212 is movable between a collapsed arrangement and an expanded arrangement with the interwoven structure moving laterally outward in the expanded arrangement. Mesh element assembly 212 can be expanded or collapsed by various means. One example involves manufacturing interwoven wires with a memory metallic alloy (e.g. Nitinol) which have a preformed expanded shape that is constrained in a catheter lumen and then allowed to recover to preformed shape upon exit of the catheter lumen. In use, the aorticorenal ganglion modifying catheter 16 containing mesh element assembly 212 is inserted into a targeted treatment site within vessel lumen 10 in the collapsed arrangement. Mesh element assembly 212 is expanded and ceases expansion once significant resistance occurs between assembly and the inner vessel lumen surface. Tissue modification with a mesh aorticorenal ganglion modifying catheter is performed similarly as described with monopolar or bipolar aorticorenal ganglion modifying catheter. It is advantageous to use a balloon 26, shown in FIG. 42, to displace the blood in proximity to the modifying element(s) 18 prior to tissue modification.

Tissue modifying element 18 and/or tissue stimulating element 31 and/or physiological measurement element 32 may also be designed as a flexible circuit assembly 216. Flexible circuits, also known as flex circuits, is a technology for mounting electronic devices on flexible plastic substrates, such as polyimide or transparent conductive polyester film which carry conductor traces. Flexible circuit assemblies 216 may be directly attached to the distal end assembly (e.g. balloon element assembly 26) of the aorticorenal ganglion modifying catheter 16 or alternatively not directly attached to the distal end assembly but attached to the distal end of the catheter elongated body 17. In the latter configuration, when the distal end assembly (e.g. mesh element assembly 212) is expanded within the renal artery, the flexible circuit assembly will be located between the distal end assembly and the renal artery wall and held in position by the circumferential expansion of the distal end assembly and its attachment to the catheter distal assembly.

### Experiment 3 Section

A chronic swine study was performed to demonstrate a reduction in renal nerve activity after percutaneous catheter modification of the aorticorenal ganglia (ARG).

Experiment 3 was similar to Experiment 1 with the exception that the ARG was modified intravascularly by percutaneous catheter RF ablation at a single site (i.e. Target Location) within the renal artery.

The study involved three treated and two naive swine. Urine and blood panel analysis were performed at each stage of the study, pre-/post-treatment and pre-kidney tissue harvest, and nephrograms were performed for the treated swine pre-treatment and pre-kidney tissue harvest. A veterinarian managed assessment of animal health throughout the study based on this data and assured proper pharmacology and diet were applied.

The procedure involved creating percutaneous access to the renal arterial vasculature through a femoral puncture site of an anesthetized test animal in dorsal recumbency. Guide catheters were placed with fluoroscopic guidance into the first renal artery. A modified electrophysiology catheter (7 French Ablatr™ Ablation Catheter, Medtronic, Minneapolis, Minn.) and a blood velocity and pressure sensing wire (Volcano CombowireXT Volcano Corp. San Diego, Calif.), were placed into the renal artery via the guide catheters.

Electrophysiology catheter was attached to an AD Instruments ML 1001 Electronic Stimulator (AD Instruments Pty Ltd, New South Wales, Australia; manufactured by Nihon Kohden Corporation, Nishiochiai, Shinjuku-ku, Tokyo, Japan) with distal tip electrode being the active electrode. Location of the ARG was determined by positioning the distal tip electrode in a longitudinal step-wise motion along the superior surface of the vessel wall while simultaneously delivering electrical stimulation (7.5 volts, 20 Hz, 7.5 msec. pulse width, biphasic). Relative proximity of the catheter tip to the aorticorenal ganglia was determined by monitoring for reduction in blood velocity and renal artery pressure using the Volcano ComboWireXT connected to a ComboMap analyzer (Volcano Corp., Rancho Cordova, Calif., USA).

After stimulation, catheter was disconnected from stimulator and connected to a Radionics RFG3 electrosurgical generator. Radiofrequency energy was delivered twice at an electrode temperature of 70° C. for a period of 60 seconds to ablate the adjacent tissue. The electrical stimulation and RF ablation procedures were repeated for the contralateral renal artery.

At 8 days, the animals were sacrificed and renal cortical samples were removed for measurement of renal cortex norepinephrine levels. Three treated animals were compared to two naïve control animals. Renal norepinephrine was reduced 74% in the treated swine compared to the controls.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for treating hypertension comprising:
advancing a treatment catheter system comprising a plurality of spaced apart tissue stimulation elements and a corresponding plurality of spaced apart tissue modification elements within a patient to a location at or near which an aorticorenal ganglion is thought to exist;
electrically stimulating said patient with a first of said plurality of spaced apart tissue stimulation elements of said treatment catheter system at said location, without resulting in a permanent change to tissue of the aorticorenal ganglion and/or a permanent change to kidney nerve activity;
detecting a first physiological response of the patient that indicates said first of said plurality of spaced apart tissue stimulation elements of said treatment catheter system is not in proximity to said aorticorenal ganglion, responsive to said stimulating when electrically stimulating said patient with said first of said plurality of spaced apart tissue stimulation elements of said treatment catheter system at said location;
electrically stimulating said patient with a second of said plurality of spaced apart tissue stimulation elements of said treatment catheter system at said location, without resulting in a permanent change to tissue of the aorticorenal ganglion or a permanent change to kidney nerve activity;
detecting a second physiological response of the patient that indicates said second of said plurality of spaced apart tissue stimulation elements of said treatment catheter system is in proximity to said aorticorenal ganglion when electrically stimulating said patient with said second of said plurality of spaced apart tissue stimulation elements of said treatment catheter system at said location; and
ablating said aorticorenal ganglion with a corresponding second of said plurality of spaced apart tissue modification elements of said treatment catheter system when detecting said first physiological response of the patient that indicates said first of said plurality of spaced apart tissue stimulation elements of said treatment catheter system is not in proximity to said aorticorenal ganglion and detecting said second physiological response of the patient that indicates said second of said plurality of spaced apart tissue stimulation elements of said treatment catheter system is in proximity to said aorticorenal ganglion, said ablating resulting in a permanent change to the tissue of the aorticorenal ganglion and/or a permanent change to kidney nerve activity.

2. The method of claim 1, wherein said detecting the second physiological response of the patient that indicates said second of said plurality of spaced apart tissue stimulation elements of determining said treatment catheter system is in proximity to said aorticorenal ganglion when electrically stimulating said patient with said second of said plurality of spaced apart tissue stimulation elements of said treatment catheter system at said location includes detecting monitoring one or more of: a change in renal blood flow velocity, renal vasoconstriction, a change in renal blood flow, and a change in renal artery blood pressure, responsive to said stimulating.

3. The method of claim 1, wherein said treatment catheter system comprises one or more catheters that may each comprise perform one or more of the following functions: stimulation, sensing said electrically stimulating, detecting, and ablating.

4. The method of claim 1, wherein said treatment catheter system further comprises one or more of: an expandable balloon, an expandable cage, a plurality of arms, or an extendable needle.

5. The method of claim 1 wherein said electric stimulation is between 0.1-100 Hz, 0.1-30 volts, and is pulsed with a pulse duration between 0.1-10 ms.

6. The method of claim 1, wherein said modifying ablating said aorticorenal ganglion further comprises applying radiofrequency energy with said treatment catheter system to said aorticorenal ganglion.

7. The method of claim 1, wherein said determining detecting the second physiological response of the patient that indicates said second of said plurality of spaced apart tissue stimulation elements of said treatment catheter system is in proximity to said aorticorenal ganglion further electrically stimulating said patient with said second of said plurality of spaced apart tissue stimulation elements of said treatment catheter system at said location comprises sensing with one or more sensors selected from the group of: an electromyogram sensor, a thermocouple, a pressure transducer, an ultrasound transducer, and an optical coherence tomography sensor.

8. The method of claim 1, wherein said advancing said treatment catheter system comprising the plurality of spaced apart tissue stimulation elements and the corresponding plurality of spaced apart tissue modification elements within said patient comprising to the location at or near which the aorticorenal ganglion is thought to exist comprises advancing said treatment catheter system within a renal artery, abdominal aorta, vena cava, renal vein, or ostia of said patient.

9. The method of claim 1, wherein said determining detecting the second physiological response of the patient that indicates said second of said plurality of spaced apart tissue stimulation elements of said treatment catheter system is in proximity to said aorticorenal ganglion when electrically stimulating said patient with said second of said plurality of spaced apart tissue stimulation elements of said treatment catheter system at said location is performed by a control box connected to said catheter treatment system.

10. A treatment system comprising:
a catheter to be inserted into positioned in a location in a patient at or near which an aorticorenal ganglion is thought to exist, the catheter comprising:
a plurality of spaced apart electrical stimulation element elements;
a sensing element; and,
an a plurality of aorticorenal ganglion modifying element ablating elements, each co-located with a corresponding one of the plurality of spaced apart electrical stimulation elements; and
a control box in electronic communication with said catheter, said control box to:
activate a first of said plurality of spaced apart electrical stimulation element elements in a pattern determined by software executed by said control box when said catheter is inserted into positioned in said location in said patient, without resulting in a permanent change to tissue of the aorticorenal ganglion and/or a permanent change to kidney nerve activity;
detect, with said sensing element, a first physiological response of said patient to said activation of said first of said plurality of spaced apart electrical stimulation elements of said catheter at said location in said patient;
determine, based on a communication with said sensing element responsive to said detection of said first physiological response activation of said stimulation element, said first of said plurality of spaced apart electrical stimulation elements of said catheter is not proximate an the aorticorenal ganglion in said patient when said first of said plurality of spaced apart electrical stimulation elements of said catheter is activated;
activate a second of said plurality of spaced apart electrical stimulation elements in a pattern determined by software executed by said control box when said catheter is positioned in said location in said patient, without resulting in a permanent change to tissue of the aorticorenal ganglion and/or a permanent change to kidney nerve activity;
detect, with said sensing element, a second physiological response of said patient to said activation of said second of said plurality of spaced apart electrical stimulation elements of said catheter at said second location of said patient;
determine, based on a communication with said sensing element responsive to said detection of said second physiological response, said second of said plurality of spaced apart electrical stimulation elements of said catheter is proximate the aorticorenal ganglion when said second of said plurality of spaced apart electrical stimulation elements of said catheter is activated; and
activate a second of said plurality of aorticorenal ganglion ablating elements corresponding to said second of said plurality of spaced apart electrical stimulation elements, said second of said plurality of aorticorenal ganglion modifying element ablating elements to modify permanently change tissue of said aorticorenal ganglion in response to said determination that said first of said plurality of spaced apart electrical stimulation elements of said catheter is not proximate the aorticorenal ganglion when said first of said plurality of spaced apart electrical stimulation elements of said catheter is activated and further in response to said determination that said second of said plurality of spaced apart electrical stimulation elements of said catheter is proximate to said aorticorenal ganglion, resulting in a permanent change to tissue of the aorticorenal ganglion and/or a permanent change of kidney nerve activity.

11. The treatment system of claim 10, wherein said catheter comprises an element selected from a group consisting of: an expandable balloon, a helical member, an expandable cage, a plurality of arms, and an extendable needle.

12. The treatment system of claim 10, wherein said control box to communicate with said sensing element to detect said first or second physiological response includes said sensing element to detect determine said catheter is proximate said aorticorenal ganglion based on one or more of the following physiological parameters to be sensed by said sensing element: a change in renal blood flow, a change in renal blood flow velocity, renal vasoconstriction, and a change in renal artery blood pressure.

13. The treatment system of claim 10, wherein each of said plurality of aorticorenal ganglion modifying element ablating elements is configured to direct aorticorenal ganglion modifying energy in a radial direction towards said aorticorenal ganglion.

14. The treatment system of claim 10, wherein each of said plurality of aorticorenal ganglion modifying element ablating elements is configured to direct aorticorenal ganglion modifying energy in an axial direction towards said aorticorenal ganglion.

15. A method of ablating an aorticorenal ganglion located in an abdominal region of a human, wherein the aorticorenal ganglion is coupled to but separate from a renal plexus, the method comprising:
inserting an apparatus into a location in a body lumen located in the abdominal region of the human near which the aorticorenal ganglion is thought to exist, the apparatus comprising a plurality of electrical stimulation element, elements, a corresponding plurality of aorticorenal ganglion ablation elements, and an aorticorenal ganglion detection element, and an aorticorenal ganglion ablation element;
activating a first of the plurality of the electrical stimulation element elements after the apparatus is inserted into the location in the body lumen, the activated first of the plurality of electrical stimulation elements to stimulate the aorticorenal ganglion when the aorticorenal ganglion is near the first of the plurality of electrical stimulation elements, without resulting in a permanent change to tissue of the aorticorenal ganglion and/or a permanent change to kidney nerve activity;
activating the aorticorenal ganglion detection element to detect a first physiological change in the human in response to activation of the first of the plurality of electrical stimulation element elements, the first physiological change indicating the aorticorenal ganglion is not near the first of the plurality of electrical stimulation elements located adjacent the body lumen;
activating a second of the plurality of electrical stimulation elements after the apparatus is inserted into the location in the body lumen, the activated second of the plurality of electrical stimulation elements to stimulate the aorticorenal ganglion when the aorticorenal ganglion is near the second of the plurality of electrical stimulation elements, without resulting in a permanent change to tissue of the aorticorenal ganglion and/or a permanent change to kidney nerve activity;
activating the aorticorenal ganglion detection element to detect a second physiological change in the human in response to activation of the second of the plurality of electrical stimulation elements, the second physiological change indicating the aorticorenal ganglion is near the second of the plurality of electrical stimulation elements; and
activating a corresponding second of the plurality of the aorticorenal ganglion ablation element elements while the apparatus remains at the location in the body lumen to ablate the aorticorenal ganglion, responsive to the aorticorenal ganglion detection element detecting the first physiological change and the second physiological change in the human, resulting in the permanent change to tissue of the aorticorenal ganglion and/or a permanent change to kidney nerve activity.

16. The method of claim 15, wherein activating the corresponding second of the plurality of co-located aorticorenal ganglion ablation element elements while the apparatus remains at the location in the body lumen to ablate the aorticorenal ganglion comprises activating the corresponding second of the plurality of co-located aorticorenal ganglion ablation element elements while the apparatus remains at the location in the body lumen to ablate the aorticorenal ganglion sufficient to cause disruption to kidney nerve activity disrupt the renal plexus.

17. The method of claim 15, wherein the body lumen is selected from a group of body lumens consisting of: a renal artery, an abdominal aorta, a vena cava, a renal vein, and ostia thereof.

18. The method of claim 15, wherein the apparatus comprising the plurality of electrical stimulation element elements, the corresponding plurality of co-located aorticorenal ganglion ablation elements, and the aorticorenal ganglion detection element, the aorticorenal ganglion ablation element, and combinations thereof, are arranged into one of a one-, two-, or three-catheter assembly.

19. The method of claim 15, wherein each of the plurality of electrical stimulation element elements and the a corresponding one of the plurality of aorticorenal ganglion ablation element elements are the same element.

20. The method of claim 15, wherein activating the aorticorenal ganglion detection element to detect a first the second physiological change in the human in response to activation of the second of the plurality of electrical stimulation element elements, wherein the first second physiological change indicates the aorticorenal ganglion is near the second of the plurality of electrical stimulation elements located adjacent the body lumen, comprises detecting one or more first physiological changes consisting of: renal vasoconstriction, decreased renal blood flow, reduced glomerular filtration rate, pulsation of a kidney, and pulsation of renal vasculature.

21. An apparatus to permanently modify tissue of an aorticorenal ganglion located in an abdominal region of a human, wherein the aorticorenal ganglion is coupled to but separate from a renal plexus, the apparatus comprising:
a catheter system to insert into a location in a body lumen located in the abdominal region of the human, near which the aorticorenal ganglion is thought to exist, the catheter system comprising:
a plurality of electrical stimulation element elements;
a physiological measurement element; and
a plurality of aorticorenal ganglion modification element elements, each co-located with a corresponding one of the plurality of electrical stimulation elements;
a control box in electronic communication with said catheter system, said control box to:
activate a first of the plurality of electrical stimulation element elements when the catheter system is inserted into the location in the body lumen, the activated first of the plurality of electrical stimulation elements to stimulate the aorticorenal ganglion when the aorticorenal ganglion is located adjacent the body lumen, near the first of the plurality of electrical stimulation elements, without resulting in a permanent modification of tissue of the aorticorenal ganglion and/or a permanent decrease of kidney nerve activity;
measure with activate the physiological measurement element to measure a first physiological response in the human in response to activation of the first of the plurality of electrical stimulation element elements, the first physiological response change indicating the aorticorenal ganglion is not near the first of the plurality of electrical stimulation elements located adjacent the body lumen; and
activate a second of the plurality of electrical stimulation elements when the catheter system is inserted into the location in the body lumen, the activated second of the plurality of electrical stimulation elements to stimulate the aorticorenal ganglion when the aorticorenal ganglion is near the second of the plurality of electrical stimulation elements, without resulting in a permanent modification of tissue of the aorticorenal ganglion and/or a permanent decrease of kidney nerve activity;
measure with the physiological measurement element a second physiological response in the human in response to activation of the second of the plurality of electrical stimulation elements, the second physiological response indicating the aorticorenal ganglion is near the second of the plurality of electrical stimulation elements;
activate a second of the plurality of aorticorenal ganglion modification element elements co-located with the second of the plurality of electrical stimulation elements, while the catheter system remains inserted into the location in the body lumen, to modify the aorticorenal ganglion in response to the physiological measurement element measuring the first second physiological response in the human, resulting in the permanent modification of tissue of the aorticorenal ganglion and/or the permanent decrease of kidney nerve activity.

22. The apparatus of claim 21, wherein the catheter system comprises one of a one-, two-, or three-catheter assembly.

23. The apparatus of claim 21, wherein each of the plurality of electrical stimulation element elements and each of the plurality of aorticorenal ganglion modification element elements that is co-located with a corresponding electrical stimulation element are the same element.

24. The apparatus of claim 21, wherein the control box to measure with activation of the physiological measurement element to measure a the first or the second physiological response in the human in response to activation of the stimulation element comprises the control box to measure with activation of the physiological measurement element to measure one or more first physiological responses changes consisting of: renal vasoconstriction, decreased renal blood flow, reduced glomerular filtration rate, pulsation of a kidney, and pulsation of renal vasculature.
